SEQUENCE LISTING (12 sequences)

SEQ ID NO: 1 - protein, 117 aa, Artificial sequence [sequence omitted]

SEQ ID NO: 2 - protein, 107 aa, Artificial sequence [sequence omitted]

SEQ ID NO: 3 - protein, 8 aa, Artificial sequence
  HCDR1
GFTFSNFG

SEQ ID NO: 4 - protein, 8 aa, Artificial sequence
  HCDR2
ISGGGRDT

SEQ ID NO: 5 - protein, 10 aa, Artificial sequence
  HCDR3
VKWGNIYFDY

SEQ ID NO: 6 - protein, 6 aa, Artificial sequence
  LCDR1
LSINTF

SEQ ID NO: 7 - protein, 3 aa, Artificial sequence
  LCDR2
AAS

SEQ ID NO: 8 - protein, 9 aa, Artificial sequence
  LCDR3
QQSSNTPFT

SEQ ID NO: 9 - protein, 444 aa, Artificial sequence
  HC 1-117; 118-444
EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 214 aa, Artificial sequence
  LC 1-108; 109-214
DIQMTQSPSSLSASVGDSITITCRASLSINTFLNWYQQKPGKAPNLLIYAASSLHGGVPSRFSGSGSGTDFTLTIRTLQPEDFATYYCQQSSNTPFTFGPGTVVDFRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 120 aa, Artificial sequence
  HCVR
EVQLVESGGGVVRPGGSLRLSCAASGFTFDDYGMTWVRQAPGRGLEWVSGIHWHGKRTGYADSVKGRFTISRDNAKKSLYLQMNSLKGEDTALYHCVRGGMSTGDWFDPWGQGTLVIVSS

SEQ ID NO: 12 - protein, 108 aa, Artificial sequence
  LCVR
DIQMTQSPSSLSASLGDRVTITCRASQSINSYLNWYQQKPGKAPKLLIYVASSLQSGVPSRFSGSGSGTEFTLTISNLQPEDFATYYCQQSYSTPPITFGQGTRLEIK

(19) United States
(12) Patent Application Publication
(10) Pub. No.: US 2023/0323470 A1
(43) Pub. Date: Oct. 12, 2023

(54) METHODS OF TREATING CANCER BY ADMINISTERING A PD-1 INHIBITOR

(71) Applicant: Regeneron Pharmaceuticals, Inc., Tarrytown, NY (US)

(72) Inventors: Matthew G. Fury, New York, NY (US); Gavin Thurston, Briarcliff Manor, NY (US); Vladimir Jankovic, New York, NY (US); Nathalie M. Fiaschi, Upper Nyack, NY (US); Israel Lowy, Dobbs Ferry, NY (US)

(21) Appl. No.: 18/042,441

(22) PCT Filed: Aug. 25, 2021
(86) PCT No.: PCT/US2021/047442

§ 371 Date: Feb. 21, 2023

Related U.S. Application Data
(60) Provisional application No. 63/094,438, filed on Oct. 21, 2020.
(60) Provisional application No. 63/070,401, filed on Aug. 26, 2020.

(51) Int. Cl. C12Q 1/6886; C07K 16/28; A61P 35/00; A61P 35/04

(52) U.S. Cl. CPC C12Q 1/6886; C07K 16/2818; A61P 35/00; A61P 35/04; C12Q 2600/156; C12Q 2600/158; C12Q 2600/106; C07K 2317/565; A61K 2039/545

(57) ABSTRACT

The present disclosure provides methods for treating or inhibiting the growth of a tumor, including selecting a patient with cancer, wherein the patient has a tumor with threshold levels of both tumor mutation burden and expression of major histocompatibility complex, and administering to the patient a therapeutically effective amount of programmed death 1 (PD-1) inhibitor (e.g., an anti-PD-1 antibody or antigen-binding fragment thereof). In some embodiments, the cancer is skin cancer, such as basal cell carcinoma or cutaneous squamous cell carcinoma.

The present disclosure generally relates to methods of treating or inhibiting the growth of a tumor, including selecting a patient with cancer in need thereof and administering to the patient a therapeutically effective amount of a programmed death 1 (PD-1) inhibitor.

### BACKGROUND

Programmed death-1 (PD-1) (also called CD279) is a 288 amino acid protein receptor expressed on activated T-cells and B-cells, natural killer cells and monocytes. PD-1 is a member of the CD28/CTLA-4 (cytotoxic T lymphocyte antigen)/ICOS (inducible co-stimulator) family of T-cell co-inhibitory receptors (Chen et al., 2013, Nat. Rev. Immunol., 13:227-242). The primary function of PD-1 is to attenuate the immune response (Riley, 2009, Immunol. Rev., 229:114-125). PD-1 has two ligands, PD-ligand 1 (PD-L1) and PD-ligand 2 (PD-L2). PD-L1 (CD274, B7H1) is widely expressed on both lymphoid and non-lymphoid tissues, such as CD4 and CD8 T-cells, macrophage lineage cells, peripheral tissues as well as on tumor cells, virally-infected cells and autoimmune tissue cells. PD-L2 (CD273, B7-DC) has a more restricted expression than PD-L1, being expressed on activated dendritic cells and macrophages (Dong et al., 1999, Nature Med., 5(12):1365-1369). PD-L1 is expressed in most human cancers, including melanoma, glioma, non-small cell lung cancer, squamous cell carcinoma of head and neck, leukemia, pancreatic cancer, renal cell carcinoma, and hepatocellular carcinoma, and may be inducible in nearly all cancer types (Zou, 2008, Nat. Rev. Immunol., 8:467-77). PD-1 binding to its ligands results in decreased T-cell proliferation and cytokine secretion, compromising humoral and cellular immune responses in diseases such as cancer, viral infection and autoimmune disease. Blockade of PD-1 binding to reverse immunosuppression has been studied in autoimmune, viral and tumor immunotherapy (Ribas 2012, NEJM 366:2517-2519; Watanabe et al., 2012, Clin. Dev. Immunol. Vol. 2012, Article ID: 269756; Wang et al., 2013, J. Viral Hep., 20:27-39).

T-cell co-stimulatory and co-inhibitory molecules (collectively named co-signaling molecules) play a crucial role in regulating T-cell activation, subset differentiation, effector function and survival (Chen et al., 2013, Nat. Rev. Immunol., 13:227-242). Following recognition of cognate peptide-MHC complexes on antigen-presenting cells by the T-cell receptor, co-signaling receptors co-localize with T-cell receptors at the immune synapse, where they synergize with T-cell receptor signaling to promote or inhibit T-cell activation and function (Flies et al., 2011, Yale J. Biol. Med., 84:409-421). The ultimate immune response is regulated by a balance between co-stimulatory and co-inhibitory signals (“immune checkpoints”) (Pardoll, 2012, Nature, 12:252-264). PD-1 functions as one such “immune checkpoint” in mediating peripheral T-cell tolerance and in avoiding autoimmunity. PD-1 binds to PD-L1 or PD-L2 and inhibits T-cell activation. The ability of PD-1 to inhibit T-cell activation is exploited by chronic viral infections and tumors to evade immune response. In chronic viral infections, PD-1 is highly expressed on virus-specific T-cells, and these T-cells become “exhausted” with loss of effector functions and proliferative capacity (Freeman, 2008, PNAS, 105:10275-10276). PD-L1 is expressed on a wide variety of tumors and studies on animal models have shown that PD-L1 on tumors inhibits T-cell activation and lysis of tumor cells and may lead to increased death of tumor-specific T-cells. The PD-1:PD-L1 system also plays an important role in induced T-regulatory (Treg) cell development and in sustaining Treg function (Francisco et al., 2010, Immunol. Rev., 236:219-242).

Since PD-1 plays an important role in autoimmunity, tumor immunity and infectious immunity, it is an ideal target for immunotherapy. Blocking PD-1 with antagonists, including monoclonal antibodies, has been studied in treatments of cancer and chronic viral infections (Sheridan 2012, Nat. Biotechnol., 30:729-730). Further, blockade of PD-1 is an effective and well tolerated approach to stimulating the immune response, and has achieved therapeutic advantage against various human cancers, including melanoma, renal cell cancer (RCC), and non-small cell lung cancer (NSCLC) (Postow et al., 2015, J Clin Oncol, 33:1974-1982).

Monoclonal antibodies to PD-1 are known in the art and have been described, for example, in U.S. Pat. Nos. 9,987,500, 8,008,449, 8,168,757, US 20110008369, US 20130017199, US 20130022595, WO 2006121168, WO 20091154335, WO 2012145493, WO 2013014668, WO 2009101611, EP 2262837, and EP 2504028. Cemiplimab, for example, is a high-affinity, fully human, hinge-stabilized IgG4P antibody directed to the PD-1 receptor that potently blocks the interaction of PD-1 with its ligands, PD-L1 and PD-L2.

Skin cancer is the most common cancer in the United States (Guy et al., 2015, Am. J. Prey. Med., 48:183-87). An estimated 5.4 million cases of non-melanoma skin cancer, including basal cell carcinoma and squamous cell carcinoma, were diagnosed in the United States in 2012 (Rogers et al., 2015, JAMA Dermatol., 151(10):1081-86). Basal cell carcinoma (BCC) is the most common skin cancer in the United States, followed by cutaneous squamous cell carcinoma (CSCC) (Karia et al., 2013, J. Am. Acad. Dermatol., 68:957-966). In fact, BCC is the most common human malignancy worldwide (Puig et al., 2015, Clin Transl Oncol, 17:497-503). Ultraviolet exposure is a major risk factor for BCC (Wu et al., 2013, Am J Epidemiol, 178:890-7). The most common clinical subtype is nodular BCC. Less common clinical subtypes are superficial, morphoeic (fibrosing), and fibroepithelial.

BCC has one of the highest mutational burdens of any human malignancy (Chalmers et al., 2017, Genome Med, 9:34; Bonilla et al., 2016, Nat Genet, 48:398-406). Tumor types with high mutational burden are generally more responsive to PD-1 blockade (McGranahan et al., 2016, Science, 351:1463-9; Rizvi et al., 2015, Science, 348:124-8; Le et al., 2017, Science, 357:409-13). The risk of BCC is 10-fold higher in solid organ transplant patients (and other groups with induced or acquired lack of cutaneous immunesurveillance), suggesting that adaptive immune responses are specifically important in this disease (Euvrard et al., 2003, N Engl J Med, 348:1681-91).

Surgery is a curative option for most BCC patients, but a small percentage of patients develop unresectable locally advanced or metastatic disease, collectively referred to as advanced BCC (Migden et al., 2018, Cancer Treat Rev, 64:1-10). Virtually all BCCs are characterized by aberrant signaling of the hedgehog signaling pathway, most commonly due to sporadic loss-of-function mutation in the gene encoding protein patched homologue (PTCH), a tumor suppressor. A PTCH mutation results in loss of patched-mediated inhibition of the G-protein coupled receptor Smoothened (SMO), thereby enhancing downstream signaling that results in uncontrolled cellular proliferation (Sekulic et al., 2016, Cell, 164:831). A small percentage of BCCs arise in the context of the autosomal dominant disorder Nevoid Basal Cell Carcinoma Syndrome (NBCCS), also known as Gorlin Syndrome, in which patients carry a germline mutation in PTCH that results in de-repression of SMO (Athar et al., 2014, Cancer Res, 74:4967-4975).

Recognition of the oncogenic role of SMO in BCC led to the development of vismodegib and sonidegib, orally available inhibitors of SMO, generally referred to as Hedgehog Inhibitors (HHIs). HHIs, such as vismodegib and sonidegib, are approved for treatment of locally advanced BCC (laBCC) or metastatic BCC (mBCC). In phase 2 studies, vismodegib and sonidegib demonstrated objective response rates (ORRs) of 30% to 60% in advanced BCC (Sekulic et al., 2012, N Engl J Med, 366:2171-9; Migden et al., 2015, Lancet Oncol, 16:716-28; Sekulic et al., 2017, BMC Cancer, 17:332; Dummer et al., 2020, Br J Dermatol, 182:1369-78). However, most patients experience disease progression on or are intolerant to HHI therapy and there is no approved second-line treatment option for these patients (Sekulic et al., 2012, N Engl J Med, 366:2171-9; Change et al., 2012, Arch Dermatol, 148:1324-5). Moreover, in addition to adverse side effects of the HHIs, it was found that for patients that progress on one HHI (vismodegib), subsequent treatment with another HHI (sonedegib) did not result in tumor inhibition (Danial et al 2016, Clin. Cancer Res. 22: 1325-29). There is no approved agent for BCC in patients who experience progression of disease on HHI therapy, or who are intolerant of prior HHI therapy.

Risk factors for CSCC include UV exposure, advanced age, and immunosuppression (Alam et al 2001, New Engl. J. Med. 344 (975-983); Madan 2010, Lancet 375: 673-685). Although the vast majority of individuals with diagnosis of CSCC or BCC have a very favorable prognosis, CSCC has a greater propensity for aggressive recurrences than BCC. Individuals diagnosed with CSCC, unlike those diagnosed with BCC, have an increased mortality compared with age-matched controls (Rees et al 2015, Int. J. Cancer 137: 878-84).

Surgical resection is the centerpiece of clinical management of CSCC. The primary goal is complete resection of cancer, and acceptable cosmetic outcome is a secondary goal. Factors associated with poor prognosis in CSCC include tumor size>2 cm, tumor depth>2mm, perineural invasion, host immunosuppression, and recurrent lesions. For the small percentage of patients who develop unresectable locally recurrent or metastatic disease, treatment options are limited. Patients may be administered post-operative radiation therapy. Chemotherapy is not an attractive option for many patients due to safety and tolerability concerns.

Cemiplimab is a high-affinity, highly potent, human, hinge-stabilized IgG4 monoclonal antibody against PD-1, approved for the treatment of patients with metastatic CSCC or locally advanced CSCC who are not candidates for curative surgery or curative radiation (Migden et al., 2018, N Engl J Med, 379:341-51; Migden et al., 2020, Lancet Oncol, 21:294-305; Rischin et al., 2020, J Immunother Cancer, 8:e000775). In the first-in-human study of cemiplimab, a durable partial response (PR) was observed in a patient with metastatic BCC (mBCC) treated with cemiplimab (Falchook et al., 2016, J Immunother Cancer, 4:70).

There is a need for safe and effective therapies for treating patients with cancer, including unresectable locally advanced BCC or metastatic BCC in patients who experience disease progression on HHI therapy, or who are intolerant of prior HHI therapy.

### SUMMARY

In one aspect, the disclosed technology relates to a method of treating or inhibiting the growth of a tumor, including: (a) selecting a patient with cancer, wherein the patient has a tumor with a tumor mutation burden (TMB) of greater than or equal to 10 mutations/Mb, and wherein the patient does not exhibit downregulated major histocompatibility complex (MHC); and (b) administering to the patient a therapeutically effective amount of programmed death 1 (PD-1) inhibitor. In some embodiments, the cancer is skin cancer selected from basal cell carcinoma (BCC), cutaneous squamous cell carcinoma (CSCC), Merkel cell carcinoma, and melanoma. In some embodiments, the cancer is BCC. In some embodiments, the cancer is metastatic BCC or unresectable locally advanced BCC. In some embodiments, at least 35% of the tumor cells are positive for MHC. In some embodiments, the MHC is MHC-I. In some embodiments, the patient experienced progression of disease on Hedgehog Inhibitor (HHI) therapy or was intolerant of prior HHI therapy.

In some embodiments, the PD-1 inhibitor is administered as a monotherapy. In some embodiments, administration of the PD-1 inhibitor promotes tumor regression, reduces tumor cell load, reduces tumor burden, and/or prevents tumor recurrence in the patient. In some embodiments, the PD-1 inhibitor is administered in combination with a second therapeutic agent or therapy selected from radiation, surgery, a cancer vaccine, imiquimod, an anti-viral agent, photodynamic therapy, HHI therapy (e.g., vismodegib, sonedegib), a PD-L1 inhibitor, a LAG3 inhibitor, a cytotoxic CTLA-4 inhibitor, GITR agonist, a TIM3 inhibitor, a BTLA inhibitor, a TIGIT inhibitor, a CD38 inhibitor, a CD47 inhibitor, an IDO inhibitor, a CD28 activator, a VEGF antagonist, an Ang2 inhibitor, a TGFβ inhibitor, an EGFR inhibitor, an antibody to a tumor-specific antigen, a vaccine, a GM-CSF, an oncolytic virus, a cytotoxin, a chemotherapeutic agent, an IL-6R inhibitor, an IL-4R inhibitor, an IL-10 inhibitor, a cytokine, an antibody drug conjugate, an anti-inflammatory drug, and a dietary supplement.

In some embodiments, the PD-1 inhibitor is selected from an anti-PD-1 antibody or antigen-binding fragment thereof, an anti-PD-L1 antibody or antigen-binding fragment thereof, and an anti-PD-L2 antibody or antigen-binding fragment thereof. In some embodiments, the PD-1 inhibitor is selected from an anti-PD-1 antibody or antigen-binding fragment thereof. In some embodiments, the PD-1 inhibitor is an anti-PD-1 antibody or antigen-binding fragment thereof that includes a heavy chain variable region (HCVR) including three heavy chain complementarity determining regions (CDRs) (HCDR1, HCDR2 and HCDR3) and a light chain variable region (LCVR) including three light chain CDRs (LCDR1, LCDR2 and LCDR3), wherein: HCDR1 has an amino acid sequence of SEQ ID NO: 3; HCDR2 has an amino acid sequence of SEQ ID NO: 4; HCDR3 has an amino acid sequence of SEQ ID NO: 5; LCDR1 has an amino acid sequence of SEQ ID NO: 6; LCDR2 has an amino acid sequence of SEQ ID NO: 7; and LCDR3 has an amino acid sequence of SEQ ID NO: 8. In some embodiments, the HCVR includes an amino acid sequence of SEQ ID NO: 1. In some embodiments, the LCVR includes an amino acid sequence of SEQ ID NO: 2. In some embodiments, the anti-PD-1 antibody or antigen-binding fragment thereof includes an HCVR/LCVR amino acid sequence pair of SEQ ID NOs: 1/2.

In some embodiments, the anti-PD-1 antibody or antigen-binding fragment thereof includes a heavy chain and a light chain, wherein the heavy chain has an amino acid sequence of SEQ ID NO: 9. In some embodiments, the anti-PD-1 antibody includes a heavy chain and a light chain, wherein the light chain has an amino acid sequence of SEQ ID NO: 10. In some embodiments, the anti-PD-1 antibody includes a heavy chain and a light chain, wherein the heavy chain has an amino acid sequence of SEQ ID NO: 9 and the light chain has an amino acid sequence of SEQ ID NO: 10.

In some embodiments, the PD-1 inhibitor is an anti-PD-1 antibody or antigen-binding fragment thereof including a HCVR with 90% sequence identity to SEQ ID NO: 1. In some embodiments, the PD-1 inhibitor is an anti-PD-1 antibody or antigen-binding fragment thereof including a LCVR with 90% sequence identity to SEQ ID NO: 2. In some embodiments, the PD-1 inhibitor is an anti-PD-1 antibody or antigen-binding fragment thereof including a HCVR with 90% sequence identity to SEQ ID NO: 1, and a LCVR with 90% sequence identity to SEQ ID NO: 2.

In some embodiments, the PD-1 inhibitor is cemiplimab or a bioequivalent thereof. In some embodiments, the PD-1 inhibitor is an anti-PD-1 antibody selected from the group consisting of cemiplimab, nivolumab, pembrolizumab, pidilizumab, MEDI0608, BI 754091, PF-06801591, spartalizumab, camrelizumab, JNJ-63723283, and MCLA-134. In some embodiments, the PD-1 inhibitor is an anti-PD-L1 antibody selected from the group consisting of REGN3504, avelumab, atezolizumab, durvalumab, MDX-1105, LY3300054, FAZ053, STI-1014, CX-072, KN035, and CK-301.

In some embodiments, the PD-1 inhibitor is administered at a dose of 5 mg to 1500 mg. In some embodiments, the PD-1 inhibitor is administered at a dose of 200 mg, 250 mg, 350 mg, 600 mg, 700 mg, or 1050 mg. In some embodiments, the PD-1 inhibitor is administered at a dose of 1 mg/kg to 20 mg/kg of the patient's body weight. In some embodiments, the PD-1 inhibitor is administered at a dose of 1 mg/kg, 3 mg/kg or 10 mg/kg of the patient's body weight. In some embodiments, the PD-1 inhibitor is administered as one or more doses, wherein each dose is administered two weeks, three weeks, four weeks, five weeks or six weeks after the immediately preceding dose. In some embodiments, the PD-1 inhibitor is administered intravenously, subcutaneously, or intraperitoneally.

In another aspect, the disclosed technology relates to a kit comprising a programmed death 1 (PD-1) inhibitor in combination with written instructions for use of a therapeutically effective amount of the PD-1 inhibitor for treating or inhibiting the growth of a tumor in a patient with cancer, wherein the patient has a tumor with a tumor mutation burden (TMB) of greater than or equal to 10 mutations/Mb, and wherein the patient does not exhibit downregulated major histocompatibility complex (MHC).

In another aspect, the disclosed technology relates to a method of treating or inhibiting the growth of a tumor, including: (a) selecting a patient with a basal cell carcinoma (BCC) tumor, wherein the patient has experienced progression of disease on Hedgehog Inhibitor (HHI) therapy or was intolerant of prior HHI therapy; (b) collecting a biopsy of the tumor; (c) measuring the tumor mutation burden (TMB) of the tumor biopsy; (d) measuring the expression of major histocompatibility complex (MHC)-I in the tumor biopsy; and (e) administering to the patient a therapeutically effective amount of programmed death 1 (PD-1) inhibitor if the tumor biopsy exhibits a TMB of greater than or equal to 10 mutations/Mb, and if at least 35% of the tumor biopsy cells are positive for MHC-I expression.

In another aspect, the disclosed technology relates to a method of selecting a patient with a basal cell carcinoma (BCC) tumor for treatment with a programmed death 1 (PD-1) inhibitor, including: (a) collecting a biopsy of the BCC tumor; (b) measuring the tumor mutation burden (TMB) of the tumor biopsy; (c) measuring the expression of major histocompatibility complex (MHC)-I in the tumor biopsy; and (d) selecting the patient for treatment with a PD-1 inhibitor if the tumor biopsy has a TMB of greater than or equal to 10 mutations/Mb, and a positive MHC-I expression in at least 35% of tumor cells.

Other embodiments of the present disclosure will become apparent from the detailed description below.

### BRIEF DESCRIPTION OF THE DRAWINGS

FIG. 1 shows swimmer plots that depict tumor response to cemiplimab, including both time to response and duration of response, in patients with locally advanced BCC (laBCC) included in the study described in Example 1 herein.

FIG. 2 is a graph showing overall survival (OS) of laBCC patients included in the study described in Example 1 herein.

FIG. 3 is a graph showing progression-free survival (PFS) of laBCC patients included in the study described in Example 1 herein.

FIG. 4 is a graph showing duration of response of laBCC patients included in the study described in Example 1 herein.

FIG. 5 is a graph showing progression free survival of laBCC patients included in the study described in Example 1 herein.

FIG. 6 is a graph showing overall survival of laBCC patients included in the study described in Example 1 herein.

FIG. 7 is a graph showing clinical activity of cemiplimab and tumor mutational burden (TMB) in laBCC patients included in the study described in Example 1 herein.

FIG. 8 is a graph showing TMB for laBCC patients who achieved durable disease control versus those who did not in connection with the study described in Example 1 herein.

FIG. 9 is a graph showing MHC-I expression in pre-treatment tumors of Responders (R) and Non-Responders (NR), including percentage of total tumor cells in laBCC patients with low (≤10 mutations/Mb) or high (>10 mutations/Mb) TMB, in connection with the study described in Example 1 herein.

FIG. 10 is a graph showing percentage of tumor cells positive for MHC-I in laBCC patients with TMB cutoff at a median TMB of 34.6 mut/Mb, in connection with the study described in Example 1 herein.

FIG. 11 shows swimmer plots that depict tumor response to cemiplimab, including both time to response and durability of responses, in patients with metastatic BCC (mBCC) included in the study described in Example 1 herein.

FIG. 12 is a graph showing a Kaplan-Meier (KM) curve for overall survival (OS) of mBCC patients included in the study described in Example 1 herein.

FIG. 13 is a graph showing a Kaplan-Meier (KM) curve for progression-free survival (PFS) of mBCC patients included in the study described in Example 1 herein.

### DETAILED DESCRIPTION

It is to be understood that the present disclosure is not limited to the particular methods and experimental conditions described, as such methods and conditions may vary. It is also to be understood that the terminology used herein is for the purpose of describing particular embodiments only, and is not intended to be limiting, and that the scope of the present disclosure will be limited only by the appended claims. Unless defined otherwise, all technical and scientific terms used herein have the same meaning as commonly understood by one of ordinary skill in the art to which this disclosure belongs. Although any methods and materials similar or equivalent to those described herein can be used in the practice or testing of the present disclosure, preferred methods and materials are now described. All publications mentioned herein are hereby incorporated by reference in their entirety unless otherwise stated.

The present disclosure generally relates to methods of treating or inhibiting the growth of a tumor, including selecting a patient with cancer in need thereof and administering to the patient a therapeutically effective amount of a programmed death 1 (PD-1) inhibitor, wherein the patient exhibits threshold levels of both tumor mutation burden (TMB) and major histocompatibility complex (MHC). TMB is a type of biomarker that reflects the number of mutations per megabase (Mb) of tumor tissue DNA. The MHC, which includes MHC class I and MHC class II genes, is another type of biomarker, which binds peptide antigens and presents them on the cell surface for recognition by T cells. As described herein, cancer patients with high TMB and regular or high levels of MHC expression are surprisingly more responsive to therapeutic treatment with a PD-1 inhibitor.

Pre-treatment tumors may be examined to determine expression of MHC-I by immunohistochemistry (IHC), and to determine TMB. As described herein, downregulation of MHC has been shown to provide a mechanism of immune evasion, even in patients with high TMB (≥10 mut/Mb). Accordingly, by specifically selecting patients with tumors that have been determined to have high TMB and regular to high levels of MHC expression, such patients can be more effectively treated with PD-1 inhibitors. In some embodiments, such patients have locally advanced BCC (laBCC). In some embodiments, administration of the PD-1 inhibitor provides an effective second-line treatment option for BCC patients who have experienced progression of disease on HHI therapy or were intolerant of prior HHI therapy. Conversely, in some embodiments, patients with tumors that do not meet the threshold requirements of high TMB and regular to high levels of MHC expression, may be treated with alternative therapies (e.g., a PD-1 inhibitor in combination with an anti-tumor therapy, such as a combination of cemiplimab and HHI therapy).

### Methods of Treating or Inhibiting Growth of Cancer

The present disclosure includes methods for treating or inhibiting the growth of a tumor comprising selecting a patient with cancer, wherein the patient exhibits threshold levels of both TMB and MHC; and administering to the patient in need thereof an antibody or antigen-binding fragment thereof that specifically binds PD-1, PD-L1, and/or PD-L2, or any other “PD-1 inhibitor” as described herein. In the present disclosure, references to particular anti-PD-1 antibodies are provided to illustrate a representative PD-1 inhibitor, and do not limit the scope of the disclosure.

As used herein, the terms “treating”, “treat”, or the like, mean to alleviate or reduce the severity of at least one symptom or indication, to eliminate the causation of symptoms either on a temporary or permanent basis, to delay or inhibit tumor growth, to reduce tumor cell load or tumor burden, to promote tumor regression, to cause tumor shrinkage, necrosis and/or disappearance, to prevent tumor recurrence, to prevent or inhibit metastasis, to inhibit metastatic tumor growth, to eliminate the need for radiation or surgery, and/or to increase duration of survival of the subject. In many embodiments, the terms “tumor”, “lesion,” “tumor lesion,” “cancer,” and “malignancy” are used interchangeably and refer to one or more cancerous growths.

As used herein, the expression “a subject in need thereof” means a human or non-human mammal that exhibits one or more symptoms or indications of cancer, and/or who has been diagnosed with cancer, including a solid tumor and who needs treatment for the same. In many embodiments, the term “subject” may be interchangeably used with the term “patient”. For example, a human subject may be diagnosed with a primary or a metastatic tumor and/or with one or more symptoms or indications including, but not limited to, unexplained weight loss, general weakness, persistent fatigue, loss of appetite, fever, night sweats, bone pain, shortness of breath, swollen abdomen, chest pain/pressure, enlargement of spleen, and elevation in the level of a cancer-related biomarker (e.g., CA125). The expression includes subjects with primary or established tumors. In specific embodiments, the expression includes human subjects that have and/or need treatment for a solid tumor, e.g., colon cancer, breast cancer, lung cancer, prostate cancer, skin cancer (e.g., BCC and CSCC), liver cancer, bone cancer, ovarian cancer, cervical cancer, pancreatic cancer, head and neck cancer, and brain cancer. The term includes subjects with primary or metastatic tumors (advanced malignancies). In certain embodiments, the expression “a subject in need thereof” includes patients with a solid tumor that is resistant to or refractory to or is inadequately controlled by prior therapy (e.g., treatment with an anti-cancer agent). For example, the expression includes subjects who have been treated with one or more lines of prior therapy such as treatment with chemotherapy (e.g., carboplatin or docetaxel). In certain embodiments, the expression “a subject in need thereof” includes patients with a solid tumor which has been treated with one or more lines of prior therapy but which has subsequently relapsed or metastasized. For example, patients with a solid tumor that may have received treatment with one or more anti-cancer agents leading to tumor regression; however, subsequently have relapsed with cancer resistant to the one or more anti-cancer agents (e.g., chemotherapy-resistant cancer, HHI-resistant cancer) are treated with the methods of the present disclosure. The expression also includes subjects with a solid tumor for which conventional anti-cancer therapy is inadvisable, for example, due to toxic side effects. For example, the expression includes patients who have received one or more cycles of HHI with toxic side effects.

In certain embodiments, the expression “a subject in need thereof” includes subjects with cancer that have a regular or elevated level of MHC expression in tumor tissue. In one embodiment, the methods of the present disclosure are used to treat patients with cancer wherein the patients are selected on the basis that they do not exhibit downregulated MHC expression in tumor tissue. In certain embodiments, the expression “downregulated MHC expression” refers to MHC expression in less than 35% of tumor cells. The expression of MHC in tumor cells is determined by assays that are known in the art, for example, by an ELISA assay or by an immunohistochemistry (IHC) assay. In certain embodiments, MHC expression is determined by quantitating RNA expression, for example, by in situ hybridization or by RT-PCR.

In certain embodiments, the expression “a subject in need thereof” includes subjects with cancer who have high tumor mutation burden (TMB). In the context of the present disclosure, high TMB refers to at least 10 mutations per megabase (Mb) of DNA from tumor cells. In some embodiments, high TMB refers to more than 10 mutations/Mb (e.g., 11, 12, 13, 14, 15, 20, 25, 30, 35, 40, 45, 50 or more mutations per Mb) in tumor cells. In one embodiment, the methods of the present disclosure are used to treat patients with cancer wherein the patients are selected on the basis of high TMB in tumor tissue of the patient. TMB may be determined by methods that are known in the art, such as by sequencing tumor DNA using a high-throughput sequence technique, e.g., next-generation sequencing (NGS) or an NGS-based method (e.g., whole genome sequencing, whole exome sequencing, or comprehensive genomic profiling of cancer gene panels). In some embodiments, TMB refers to the number of nonsynonymous mutations per megabase of DNA sequenced.

In certain preferred embodiments, the expression “a subject in need thereof” includes subjects with cancer who have high TMB and do not exhibit downregulated MHC expression in tumor tissue. In one embodiment, the methods of the present disclosure are used to treat patients with cancer, wherein the patients are selected on the basis that they have high TMB and do not exhibit downregulated MHC expression in tumor tissue.

In certain embodiments, the methods of the present disclosure may be used to treat patients that show elevated levels of one or more cancer-associated biomarkers (e.g., PD-L1, CA125, CA19-9, prostate-specific antigen (PSA), lactate dehydrogenase, KIT, carcinoembryonic antigen, epidermal growth factor receptor (EGFR), ALK gene rearrangement). In certain embodiments, the methods of the present disclosure are used to treat patients with a cancer wherein the patients are selected on the basis of at least 1%, at least 2%, at least 5%, at least 10%, at least 20%, at least 30%, at least 40% or at least 50% PD-L1 expression in cancer tissue and/or immune cells. Methods to determine PD-L1 expression in cancer tissue and/or immune cells are well-known in the art. In certain embodiments, the expression of PD-L1 in tumor tissue is determined by any assay known in the art, for example, by an ELISA assay or by an immunohistochemistry (IHC) assay. See, e.g., WO 2016124558; WO 2016191751; US 20160305947. In certain embodiments, the expression of PD-L1 is determined by quantitating RNA expression, for example, by in situ hybridization or by RT-PCR. In certain embodiments, the expression of PD-L1 is determined by imaging with a labeled anti-PD-L1 antibody, for example, by immuno-positron emission tomography or iPET. See, e.g., van Dongen et al., Oncologist, 12(12):1379-89 (2007); Boerman et al., J Nucl Med, 52:1171-72 (2011); US 20180161464.

In certain embodiments, the methods of the present disclosure are used in a subject with a solid tumor. As used herein, the term “solid tumor” refers to an abnormal mass of tissue that usually does not contain cysts or liquid areas. Solid tumors may be benign (not cancer) or malignant (cancer). For the purposes of the present disclosure, the term “solid tumor” means malignant solid tumor. The term includes different types of solid tumors named for the cell types that form them, viz. sarcomas, carcinomas and lymphomas. However, the term does not include leukemias. In various embodiments, the term ‘solid tumor” includes cancers arising from connective or supporting tissue (e.g., bone or muscle) (referred to as sarcomas), cancers arising from the body's glandular cells and epithelial cells which line body tissues (referred to as carcinomas), and cancers of the lymphoid organs such as lymph nodes, spleen and thymus (referred to as lymphomas). Lymphoid cells occur in almost all tissues of the body and therefore, lymphomas may develop in a wide variety of organs. In certain embodiments, the term “solid tumor” includes cancers including, but not limited to, BCC, CSCC, colorectal cancer, ovarian cancer, prostate cancer, breast cancer, brain cancer, cervical cancer, bladder cancer, anal cancer, uterine cancer, colon cancer, liver cancer, pancreatic cancer, lung cancer, endometrial cancer, bone cancer, testicular cancer, skin cancer, kidney cancer, stomach cancer, esophageal cancer, head and neck cancer, salivary gland cancer, and myeloma. In certain embodiments, the term “solid tumor” includes cancers including, but not limited to, hepatocellular carcinoma, non-small cell lung cancer, head and neck squamous cell cancer, basal cell carcinoma, breast carcinoma, cutaneous squamous cell carcinoma, chondrosarcoma, angiosarcoma, cholangiocarcinoma, soft tissue sarcoma, colorectal cancer, melanoma, Merkel cell carcinoma, and glioblastoma multiforme. In certain embodiments, the term “solid tumor” comprises more than one solid tumor lesions located separate from one another, e.g., 2, more than 2, more than 5, more than 10, more than 15, more than 20, or more than 25 lesions in a subject in need of treatment. In certain embodiments, the more than one lesions are located distally from one another in the same organ. In certain other embodiments, the tumor lesions may be located in different organs.

In certain embodiments, the present disclosure includes methods to treat or inhibit growth of a cancer including, but not limited to, colorectal cancer, ovarian cancer, prostate cancer, breast cancer, brain cancer, cervical cancer, bladder cancer, anal cancer, uterine cancer, colon cancer, liver cancer, pancreatic cancer, lung cancer, endometrial cancer, bone cancer, testicular cancer, skin cancer (BCC and CSCC), kidney cancer, stomach cancer, esophageal cancer, head and neck cancer, salivary gland cancer, and myeloma. In certain embodiments, the present disclosure includes methods to treat or inhibit the growth of a skin cancer including, but not limited to, BCC and CSCC. In one embodiment, the subject has high tumor mutation burden (≥10 mutations/Mb). In one embodiment, the subject does not exhibit downregulated MHC expression. In one embodiment, the subject has high tumor mutation burden (≥10 mutations/Mb) and does not exhibit downregulated MHC expression.

In certain embodiments, the present disclosure includes methods to treat advanced solid tumors including but not limited to, metastatic BCC, locally advanced BCC, metastatic CSCC, locally advanced CSCC, and any advanced solid tumor refractory to first-line therapy. The methods, according to this aspect, comprise selecting a patient with cancer, and administering a therapeutically effective amount of a PD-1 inhibitor (e.g., an anti-PD-1 antibody or antigen-binding fragment thereof). In one embodiment, the subject has high tumor mutation burden (≥10 mutations/Mb). In one embodiment, the subject does not exhibit downregulated MHC expression. In one embodiment, the subject has high tumor mutation burden (≥10 mutations/Mb) and does not exhibit downregulated MHC expression.

In certain embodiments, the methods comprise administering a therapeutically effective amount of a PD-1 inhibitor in combination with an anti-tumor therapy. Anti-tumor therapies include, but are not limited to, conventional anti-tumor therapies such as chemotherapy, radiation, surgery, and others described elsewhere herein. In one embodiment, the anti-tumor therapy comprises radiation therapy. In certain embodiments, one or more doses of a PD-1 inhibitor are administered to a subject in need thereof, wherein each dose is administered 0.5, 1, 2, 3, 4, 5, 6, 7, 8, 9 or 10 weeks after the immediately preceding dose.

The methods of the present disclosure, according to certain embodiments, include administering to a subject a therapeutically effective amount of a PD-1 inhibitor (e.g., an anti-PD-1 antibody or antigen-binding fragment thereof) in combination with a second therapeutic agent or therapy. The second therapeutic agent or therapy may be administered for increasing anti-tumor efficacy, for reducing toxic effects of one or more therapies and/or for reducing the dosage of one or more therapies. In various embodiments, the second therapeutic agent or therapy may include one or more of: radiation, surgery, a cancer vaccine, imiquimod, an anti-viral agent (e.g., cidofovir), photodynamic therapy, HHI therapy (e.g., vismodegib, sonedegib), a programmed death ligand 1 (PD-L1) inhibitor (e.g., an anti-PD-L1 antibody), a lymphocyte activation gene 3 (LAG3) inhibitor (e.g., an anti-LAG3 antibody), a cytotoxic T-lymphocyte-associated protein 4 (CTLA-4) inhibitor (e.g., ipilimumab), a glucocorticoid-induced tumor necrosis factor receptor (GITR) agonist (e.g., an anti-GITR antibody), a T-cell immunoglobulin and mucin containing -3 (TIM3) inhibitor, a B- and T-lymphocyte attenuator (BTLA) inhibitor, a T-cell immunoreceptor with Ig and ITIM domains (TIGIT) inhibitor, a CD38 inhibitor, a CD47 inhibitor, an indoleamine-2,3-dioxygenase (IDO) inhibitor, a CD28 activator, a vascular endothelial growth factor (VEGF) antagonist [e.g., a “VEGF-Trap” such as aflibercept, or an anti-VEGF antibody or antigen binding fragment thereof (e.g., bevacizumab, or ranibizumab) or a small molecule kinase inhibitor of VEGF receptor (e.g., sunitinib, sorafenib, or pazopanib)], an angiopoietin-2 (Ang2) inhibitor, a transforming growth factor beta (TGFβ) inhibitor, an epidermal growth factor receptor (EGFR) inhibitor, an antibody to a tumor-specific antigen [e.g., CA9, CA125, melanoma-associated antigen 3 (MAGE3), carcinoembryonic antigen (CEA), vimentin, tumor-M2-PK, prostate-specific antigen (PSA), mucin-1, MART-1, and CA19-9], a vaccine (e.g., Bacillus Calmette-Guerin), granulocyte-macrophage colony-stimulating factor (GM-CSF), an oncolytic virus, a cytotoxin, a chemotherapeutic agent (e.g., pemetrexed, dacarbazine, temozolomide, cyclophosphamide, docetaxel, doxorubicin, daunorubicin, cisplatin, carboplatin, gemcitabine, methotrexate, mitoxantrone, oxaliplatin, paclitaxel, topotecan, irinotecan, vinorelbine, and vincristine), vismodegib, sonedegib, an IL-6R inhibitor, an IL-4R inhibitor, an IL-10 inhibitor, a cytokine such as IL-2, IL-7, IL-12, IL-21, and IL-15, an antibody drug conjugate, an anti-inflammatory drug such as a corticosteroid, a non-steroidal anti-inflammatory drug (NSAID), and a dietary supplement such as an antioxidant.

In certain embodiments, the present disclosure includes methods to treat a cancer or inhibit the growth of a cancer with microsatellite instability (MSI). As used herein, the term “microsatellite instability,” also known as “MSI” refers to the changes in microsatellite repeats in tumor cells or genetic hypermutability caused due to deficient DNA mismatch repair. Microsatellites, also known as simple sequence repeats, are repeated sequences of DNA comprising repeating units 1-6 base pairs in length. Although the length of microsatellites is highly variable from person to person and contributes to the DNA fingerprint, each individual has microsatellites of a set length. MSI results from the inability of the mismatch repair (MMR) proteins to fix a DNA replication error. MSI comprises DNA polymorphisms, wherein the replication errors vary in length instead of sequence. MSI comprises frame-shift mutations, either through insertions or deletions, or hypermethylation, leading to gene silencing. It is known in the art that microsatellite instability may result in colon cancer, gastric cancer, endometrium cancer, ovarian cancer, hepatobiliary tract cancer, urinary tract cancer, brain cancer, and skin cancers. The present disclosure includes methods to treat cancers with MSI, the methods comprising administering to a patient in need thereof a therapeutically effective amount of a PD-1 inhibitor (e.g., an anti-PD-1 antibody or antigen-binding fragment thereof).

According to certain embodiments, the present disclosure includes methods for treating, or delaying or inhibiting the growth of a tumor. In certain embodiments, the present disclosure includes methods to promote tumor regression. In certain embodiments, the present disclosure includes methods to reduce tumor cell load or to reduce tumor burden. In certain embodiments, the present disclosure includes methods to prevent tumor recurrence.

In certain embodiments, the methods of the present disclosure comprise administering a therapeutically effective amount of a PD-1 inhibitor (e.g., an anti-PD-1 antibody or antigen-binding fragment thereof) to a subject with advanced solid tumors. In one embodiment, the advanced solid tumor is skin cancer. In certain other embodiments, the advanced solid tumor is BCC or CSCC. In one embodiment, the subject is not responsive to prior therapy or has relapsed after prior therapy (e.g., an HHI). In one embodiment, the subject has an advanced solid tumor that is refractory to first line chemotherapy. In one embodiment, the subject has high tumor mutation burden (≥10 mutations/Mb). In one embodiment, the subject does not exhibit downregulated MHC expression. In one embodiment, the subject has high tumor mutation burden (≥10 mutations/Mb) and does not exhibit downregulated MHC expression.

In certain embodiments, the methods of the present disclosure comprise administering a therapeutically effective amount of a PD-1 inhibitor (e.g., an anti-PD-1 antibody or antigen-binding fragment thereof) to a patient with metastatic BCC or unresectable locally advanced BCC, wherein the patient has experienced progression of disease on HHI therapy, or were intolerant of prior HHI therapy. In one embodiment, the subject has high tumor mutation burden (≥10 mutations/Mb). In one embodiment, the subject does not exhibit downregulated MHC expression. In one embodiment, the subject has high tumor mutation burden (≥10 mutations/Mb) and does not exhibit downregulated MHC expression.

According to one aspect, the present disclosure includes methods to treat or inhibit the growth of a tumor, the methods comprising: (a) selecting a patient with basal cell carcinoma (BCC) wherein the patient is selected based on one or more of the following attributes: (i) the patient has locally advanced BCC; (ii) the patient has metastatic BCC; (iii) the tumor is unresectable; (iv) the patient has been earlier treated with at least one anti-tumor therapy; (v) the patient has been treated earlier and the patient's BCC progressed upon treatment with a Hedgehog pathway inhibitor (HHI) (e.g., vismodegib, sonedegib); (vi) the patient is intolerant to a HHI (vii) the patient has disease that is considered inoperable or is not amenable to curative surgery; (viii) surgery and/or radiation is contraindicated; (ix) the patient has been earlier treated with radiation and the tumor is resistant or unresponsive to radiation; (viii) the patient shows ≥1%, ≥5%, or ≥10% PD-L1 expression in tumor cells; (ix) the tumor comprises UV-induced DNA damage; (x) the patient has high tumor mutation burden; and (xi) the patient does not exhibit downregulated MHC expression; and (b) administering a therapeutically effective amount of a PD-1 inhibitor (e.g., an anti-PD-1 antibody or antigen-binding fragment thereof) to the patient in need thereof.

One embodiment of the disclosure pertains to administration of a PD-1 inhibitor (e.g., an anti-PD-1 antibody or antigen-binding fragment thereof) for use in the treatment of advanced solid tumors in patients that have been previously treated with another anti-tumor therapy, such as HHI. One embodiment of the disclosure pertains to administration of a PD-1 inhibitor (e.g., an anti-PD-1 antibody or antigen-binding fragment thereof) for use in the treatment of advanced solid tumors that are refractory to first-line chemotherapy.

In certain embodiments, the methods of the present disclosure comprise administering to a subject in need thereof a therapeutically effective amount of a PD-1 inhibitor (e.g., an anti-PD-1 antibody or antigen-binding fragment thereof), wherein administration of the PD-1 inhibitor leads to increased overall survival (OS) or progression-free survival (PFS) of the patient as compared to a patient administered with a ‘standard-of-care’ (SOC) therapy (e.g., chemotherapy, surgery or radiation). In certain embodiments, the PFS is increased by at least one month, at least 2 months, at least 3 months, at least 4 months, at least 5 months, at least 6 months, at least 7 months, at least 8 months, at least 9 months, at least 10 months, at least 11 months, at least 1 year, at least 2 years, or at least 3 years as compared to a patient administered with any one or more SOC therapies. In certain embodiments, the OS is increased by at least one month, at least 2 months, at least 3 months, at least 4 months, at least 5 months, at least 6 months, at least 7 months, at least 8 months, at least 9 months, at least 10 months, at least 11 months, at least 1 year, at least 2 years, or at least 3 years as compared to a patient administered with any one or more SOC therapies.

In certain embodiments, the methods of the present disclosure comprise administering to a subject in need thereof a therapeutically effective amount of a PD-1 inhibitor (e.g., an anti-PD-1 antibody or antigen-binding fragment thereof), wherein administration of the PD-1 inhibitor leads to increased overall survival (OS) or progression-free survival (PFS) of the patient as compared to a patient that exhibits downregulated MHC expression (e.g., less than 35% of tumor cells are positive for MHC) and low TMB (e.g., less than 10 mut/Mb). In certain embodiments, the PFS is increased by at least one month, at least 2 months, at least 3 months, at least 4 months, at least 5 months, at least 6 months, at least 7 months, at least 8 months, at least 9 months, at least 10 months, at least 11 months, at least 1 year, at least 2 years, or at least 3 years as compared to the patient with downregulated MHC and low TMB. In certain embodiments, the OS is increased by at least one month, at least 2 months, at least 3 months, at least 4 months, at least 5 months, at least 6 months, at least 7 months, at least 8 months, at least 9 months, at least 10 months, at least 11 months, at least 1 year, at least 2 years, or at least 3 years as compared to the patient with downregulated MHC and low TMB.

The present disclosure also provides kits comprising a PD-1 inhibitor (e.g., an anti-PD-1 antibody or antigen-binding fragment thereof) for therapeutic uses as described herein. Kits typically include a label indicating the intended use of the contents of the kit and instructions for use. As used herein, the term “label” includes any writing, or recorded material supplied on, in or with the kit, or which otherwise accompanies the kit. Accordingly, this disclosure provides a kit for treating a subject afflicted with a cancer, the kit comprising: (a) a therapeutically effective dosage of a PD-1 inhibitor antibody; and (b) instructions for using the PD-1 inhibitor in any of the methods disclosed herein. In certain embodiments for treating human patients, the kit comprises a PD-1 inhibitor disclosed herein, e.g., cemiplimab, nivolumab, or pembrolizumab. In some embodiments, the instructions include collecting a tumor biopsy of the patient, determining the level of TMB and MHC expression in the tumor biopsy, and administering the PD-1 inhibitor if the tumor biopsy has a TMB of greater than or equal to 10 mut/Mb and expression of MHC in at least 35% of tumor cells.

### PD-1 Inhibitors

The methods disclosed herein include administering a therapeutically effective amount of a PD-1 inhibitor. As used herein, a “PD-1 inhibitor” refers to any molecule capable of inhibiting, blocking, abrogating or interfering with the activity or expression of PD-1. In some embodiments, the PD-1 inhibitor can be an antibody, a small molecule compound, a nucleic acid, a polypeptide, or a functional fragment or variant thereof. Non-limiting examples of suitable PD-1 inhibitor antibodies include anti-PD-1 antibodies and antigen-binding fragments thereof, anti-PD-L1 antibodies and antigen-binding fragments thereof, and anti-PD-L2 antibodies and antigen-binding fragments thereof. Other non-limiting examples of suitable PD-1 inhibitors include RNAi molecules such as anti-PD-1 RNAi molecules, anti-PD-L1 RNAi, and an anti-PD-L2 RNAi, antisense molecules such as anti-PD-1 antisense RNA, anti-PD-L1 antisense RNA, and anti-PD-L2 antisense RNA, and dominant negative proteins such as a dominant negative PD-1 protein, a dominant negative PD-L1 protein, and a dominant negative PD-L2 protein. Some examples of the foregoing PD-1 inhibitors are described in e.g., U.S. Pat. Nos. 9,308,236, 10,011,656, and US 20170290808, the portions of which that identify PD-1 inhibitors are hereby incorporated by reference.

The term “antibody,” as used herein, is intended to refer to immunoglobulin molecules comprised of four polypeptide chains, two heavy (H) chains and two light (L) chains inter-connected by disulfide bonds (i.e., “full antibody molecules”), as well as multimers thereof (e.g. IgM) or antigen-binding fragments thereof. Each heavy chain is comprised of a heavy chain variable region (“HCVR” or “V_{H}”) and a heavy chain constant region (comprised of domains CH1, CH2 and CH3). Each light chain is comprised of a light chain variable region (“LCVR or “V_{L}”) and a light chain constant region (C_{L}). The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In certain embodiments, the FRs of the antibody (or antigen binding fragment thereof) may be identical to the human germline sequences or may be naturally or artificially modified. An amino acid consensus sequence may be defined based on a side-by-side analysis of two or more CDRs. The term “antibody,” as used herein, also includes antigen-binding fragments of full antibody molecules.

As used herein, the terms “antigen-binding fragment” of an antibody, “antigen-binding portion” of an antibody, and the like, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present disclosure include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present disclosure may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

The antibodies used in the methods disclosed herein may be human antibodies. As used herein, the term “human antibody” refers to antibodies having variable and constant regions derived from human germline immunoglobulin sequences. The human antibodies of the present disclosure may nonetheless include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences.

The antibodies used in the methods disclosed herein may be recombinant human antibodies. As used herein, the term “recombinant human antibody” includes all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al., 1992, Nucl. Acids Res., 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

### Anti-PD-1 Antibodies and Antigen-Binding Fragments Thereof

In some embodiments, PD-1 inhibitors used in the methods disclosed herein are antibodies or antigen-binding fragments thereof that specifically bind PD-1. The term “specifically binds,” or the like, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. For example, an antibody that “specifically binds” PD-1, as used in the context of the present disclosure, includes antibodies that bind PD-1 or a portion thereof with a K_{D} of less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, less than about 2 nM, less than about 1 nM or less than about 0.5 nM, as measured in a surface plasmon resonance assay. An isolated antibody that specifically binds human PD-1 may, however, have cross-reactivity to other antigens, such as PD-1 molecules from other (non-human) species.

According to certain exemplary embodiments, the anti-PD-1 antibody, or antigen-binding fragment thereof comprises a heavy chain variable region (HCVR), light chain variable region (LCVR), and/or complementarity determining regions (CDRs) comprising the amino acid sequences of any of the anti-PD-1 antibodies set forth in U.S. Pat. No. 9,987,500, which is hereby incorporated by reference in its entirety. In certain exemplary embodiments, the anti-PD-1 antibody or antigen-binding fragment thereof that can be used in the context of the present disclosure comprises the heavy chain complementarity determining regions (HCDRs) of a heavy chain variable region (HCVR) comprising the amino acid sequence of SEQ ID NO: 1 and the light chain complementarity determining regions (LCDRs) of a light chain variable region (LCVR) comprising the amino acid sequence of SEQ ID NO: 2. According to certain embodiments, the anti-PD-1 antibody or antigen-binding fragment thereof comprises three HCDRs (HCDR1, HCDR2 and HCDR3) and three LCDRs (LCDR1, LCDR2 and LCDR3), wherein the HCDR1 comprises the amino acid sequence of SEQ ID NO: 3; the HCDR2 comprises the amino acid sequence of SEQ ID NO: 4; the HCDR3 comprises the amino acid sequence of SEQ ID NO: 5; the LCDR1 comprises the amino acid sequence of SEQ ID NO: 6; the LCDR2 comprises the amino acid sequence of SEQ ID NO: 7; and the LCDR3 comprises the amino acid sequence of SEQ ID NO: 8. In yet other embodiments, the anti-PD-1 antibody or antigen-binding fragment thereof comprises an HCVR comprising SEQ ID NO: 1 and an LCVR comprising SEQ ID NO: 2. In certain embodiments, the methods of the present disclosure comprise the use of an anti-PD-1 antibody, wherein the antibody comprises a heavy chain comprising the amino acid sequence of SEQ ID NO: 9. In some embodiments, the anti-PD-1 antibody comprises a light chain comprising the amino acid sequence of SEQ ID NO: 10. An exemplary antibody comprising a heavy chain variable region comprising the amino acid sequence of SEQ ID NO: 1 and a light chain variable region comprising the amino acid sequence of SEQ ID NO: 2 is the fully human anti-PD-1 antibody known as cemiplimab (also known as REGN2810; LIBTAYO®).

According to certain exemplary embodiments, the methods of the present disclosure comprise the use of cemiplimab or a bioequivalent thereof. As used herein, the term “bioequivalent” refers to anti-PD-1 antibodies or PD-1-binding proteins or fragments thereof that are pharmaceutical equivalents or pharmaceutical alternatives whose rate and/or extent of absorption do not show a significant difference with that of a reference antibody (e.g., cemiplimab) when administered at the same molar dose under similar experimental conditions, either single dose or multiple dose. In the context of the present disclosure, the term “bioequivalent” includes antigen-binding proteins that bind to PD-1 and do not have clinically meaningful differences with cemiplimab with respect to safety, purity and/or potency.

According to certain embodiments of the present disclosure, the anti-human PD-1, or antigen-binding fragment thereof, comprises a HCVR having 90%, 95%, 98% or 99% sequence identity to SEQ ID NO: 1.

According to certain embodiments of the present disclosure, the anti-human PD-1, or antigen-binding fragment thereof, comprises a LCVR having 90%, 95%, 98% or 99% sequence identity to SEQ ID NO: 2.

According to certain embodiments of the present disclosure, the anti-human PD-1, or antigen-binding fragment thereof, comprises a HCVR comprising an amino acid sequence of SEQ ID NO: 1 having no more than 5 amino acid substitutions. According to certain embodiments of the present disclosure, the anti-human PD-1, or antigen-binding fragment thereof, comprises a LCVR comprising an amino acid sequence of SEQ ID NO: 2 having no more than 2 amino acid substitutions.

Sequence identity may be measured by methods known in the art (e.g., GAP, BESTFIT, and BLAST).

The present disclosure also includes use of anti-PD-1 antibodies or antigen-binding fragments thereof in methods to treat cancer, wherein the anti-PD-1 antibodies or antigen-binding fragments thereof comprise variants of any of the HCVR, LCVR and/or CDR amino acid sequences disclosed herein having one or more conservative amino acid substitutions. For example, the present disclosure includes use of anti-PD-1 antibodies or antigen-binding fragments thereof having HCVR, LCVR and/or CDR amino acid sequences with, e.g., 10 or fewer, 8 or fewer, 6 or fewer, 4 or fewer, etc. conservative amino acid substitutions relative to any of the HCVR, LCVR and/or CDR amino acid sequences disclosed herein.

Other anti-PD-1 antibodies or antigen-binding fragments thereof that can be used in the context of the methods of the present disclosure include, e.g., the antibodies referred to and known in the art as nivolumab, pembrolizumab, MEDI0608, pidilizumab, BI 754091, spartalizumab (also known as PDR001), camrelizumab (also known as SHR-1210), JNJ-63723283, MCLA-134, or any of the anti-PD-1 antibodies set forth in U.S. Pat. Nos. 6,808,710, 7,488,802, 8,008,449, 8,168,757, 8,354,509, 8,609,089, 8,686,119, 8,779,105, 8,900,587, and 9,987,500, and in patent publications WO 2006/121168, WO 2009/114335. The portions of all of the aforementioned publications that identify anti-PD-1 antibodies are hereby incorporated by reference.

The anti-PD-1 antibodies used in the context of the methods of the present disclosure may have pH-dependent binding characteristics. For example, an anti-PD-1 antibody for use in the methods of the present disclosure may exhibit reduced binding to PD-1 at acidic pH as compared to neutral pH. Alternatively, an anti-PD-1 antibody of the invention may exhibit enhanced binding to its antigen at acidic pH as compared to neutral pH. The expression “acidic pH” includes pH values less than about 6.2, e.g., about 6.0, 5.95, 5.9, 5.85, 5.8, 5.75, 5.7, 5.65, 5.6, 5.55, 5.5, 5.45, 5.4, 5.35, 5.3, 5.25, 5.2, 5.15, 5.1, 5.05, 5.0, or less. As used herein, the expression “neutral pH” means a pH of about 7.0 to about 7.4. The expression “neutral pH” includes pH values of about 7.0, 7.05, 7.1, 7.15, 7.2, 7.25, 7.3, 7.35, and 7.4.

In certain instances, “reduced binding to PD-1 at acidic pH as compared to neutral pH” is expressed in terms of a ratio of the K_{D} value of the antibody binding to PD-1 at acidic pH to the K_{D} value of the antibody binding to PD-1 at neutral pH (or vice versa). For example, an antibody or antigen-binding fragment thereof may be regarded as exhibiting “reduced binding to PD-1 at acidic pH as compared to neutral pH” for purposes of the present disclosure if the antibody or antigen-binding fragment thereof exhibits an acidic/neutral K_{D} ratio of about 3.0 or greater. In certain exemplary embodiments, the acidic/neutral K_{D} ratio for an antibody or antigen-binding fragment of the present disclosure can be about 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 20.0, 25.0, 30.0, 40.0, 50.0, 60.0, 70.0, 100.0, or greater.

Antibodies with pH-dependent binding characteristics may be obtained, e.g., by screening a population of antibodies for reduced (or enhanced) binding to a particular antigen at acidic pH as compared to neutral pH. Additionally, modifications of the antigen-binding domain at the amino acid level may yield antibodies with pH-dependent characteristics. For example, by substituting one or more amino acids of an antigen-binding domain (e.g., within a CDR) with a histidine residue, an antibody with reduced antigen-binding at acidic pH relative to neutral pH may be obtained. As used herein, the expression “acidic pH” means a pH of 6.0 or less.

### Anti-PD-L1 Antibodies and Antigen-Binding Fragments Thereof

In some embodiments, PD-1 inhibitors used in the methods disclosed herein are antibodies or antigen-binding fragments thereof that specifically bind PD-L1. For example, an antibody that “specifically binds” PD-L1, as used in the context of the present disclosure, includes antibodies that bind PD-L1 or a portion thereof with a K_{D} of about 1×10⁻⁸ M or less (e.g., a smaller K_{D} denotes a tighter binding). A “high affinity” anti-PD-L1 antibody refers to those mAbs having a binding affinity to PD-L1, expressed as K_{D} of at least 10⁻⁸ M, preferably 10⁻⁹ M, more preferably 10⁻¹⁰ M, even more preferably 10⁻¹¹ M, even more preferably 10⁻¹² M, as measured by surface plasmon resonance, e.g., BIACORE™ or solution-affinity ELISA. An isolated antibody that specifically binds human PD-L1 may, however, have cross-reactivity to other antigens, such as PD-L1 molecules from other (non-human) species.

According to certain exemplary embodiments, the anti-PD-L1 antibody or antigen-binding fragment thereof comprises a heavy chain variable region (HCVR), light chain variable region (LCVR), and/or complementarity determining regions (CDRs) comprising the amino acid sequences of any of the anti-PD-L1 antibodies set forth in U.S. Pat. No. 9,938,345, which is hereby incorporated by reference in its entirety. In certain exemplary embodiments, an anti-PD-L1 antibody or antigen-binding fragment thereof that can be used in the context of the present disclosure comprises the heavy chain complementarity determining regions (HCDRs) of a heavy chain variable region (HCVR) comprising SEQ ID NO: 11 and the light chain complementarity determining regions (LCDRs) of a light chain variable region (LCVR) comprising SEQ ID NO: 12. An exemplary anti-PD-L1 antibody comprising a HCVR of SEQ ID NO: 11 and a LCVR of SEQ ID NO: 12 is REGN3504.

According to certain embodiments of the present disclosure, the anti-human PD-L1 antibody, or antigen-binding fragment thereof, comprises a HCVR having 90%, 95%, 98% or 99% sequence identity to SEQ ID NO: 11. According to certain embodiments of the present disclosure, the anti-human PD-L1 antibody, or antigen-binding fragment thereof, comprises a LCVR having 90%, 95%, 98% or 99% sequence identity to SEQ ID NO: 12.

According to certain embodiments of the present disclosure, the anti-human PD-L1 antibody, or antigen-binding fragment thereof, comprises a HCVR comprising an amino acid sequence of SEQ ID NO: 11 having no more than 5 amino acid substitutions. According to certain embodiments of the present disclosure, the anti-human PD-L1 antibody, or antigen-binding fragment thereof, comprises a LCVR comprising an amino acid sequence of SEQ ID NO: 12 having no more than 2 amino acid substitutions.

Sequence identity may be measured by methods known in the art (e.g., GAP, BESTFIT, and BLAST).

The present disclosure also includes use of anti-PD-L1 antibodies in methods to treat cancer, wherein the anti-PD-L1 antibodies comprise variants of any of the HCVR, LCVR and/or CDR amino acid sequences disclosed herein having one or more conservative amino acid substitutions. For example, the present disclosure includes use of anti-PD-L1 antibodies having HCVR, LCVR and/or CDR amino acid sequences with, e.g., 10 or fewer, 8 or fewer, 6 or fewer, 4 or fewer, etc. conservative amino acid substitutions relative to any of the HCVR, LCVR and/or CDR amino acid sequences disclosed herein.

Other anti-PD-L1 antibodies that can be used in the context of the methods of the present disclosure include, e.g., the antibodies referred to and known in the art as MDX-1105, atezolizumab (TECENTRIQ™), durvalumab (IMFINZI™), avelumab (BAVENCIO™), LY3300054, FAZ053, STI-1014, CX-072, KN035 (Zhang et al., Cell Discovery, 3, 170004 (March 2017)), CK-301 (Gorelik et al., American Association for Cancer Research Annual Meeting (AACR), 2016-04-04 Abstract 4606), or any of the other anti-PD-L1 antibodies set forth in patent publications U.S. Pat. Nos. 7,943,743, 8,217,149, 9,402,899, 9,624,298, 9,938,345, WO 2007/005874, WO 2010/077634, WO 2013/181452, WO 2013/181634, WO 2016/149201, WO 2017/034916, or EP3177649. The portions of all of the aforementioned publications that identify anti-PD-L1 antibodies are hereby incorporated by reference.

### Pharmaceutical Compositions and Administration

The present disclosure provides therapeutic pharmaceutical compositions comprising the PD-1 inhibitors disclosed herein. Such pharmaceutical compositions may be formulated with suitable pharmaceutically acceptable carriers, excipients, buffers, and other agents that provide suitable transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al., “Compendium of excipients for parenteral formulations” PDA, J Pharm Sci Technol 52:238-311 (1998).

The dose of PD-1 inhibitor (e.g., anti-PD-1 antibody or antigen-binding fragment thereof) may vary depending upon the age and the size of a subject to be administered, target disease, conditions, route of administration, and the like. When a PD-1 inhibitor of the present disclosure is used for treating or inhibiting the growth of cancer, it may be advantageous to administer the PD-1 inhibitor at a single dose of about 0.1 to about 100 mg/kg body weight. Depending on the severity of the condition, the frequency and the duration of the treatment can be adjusted. In certain embodiments, the PD-1 inhibitor of the present disclosure can be administered as an initial dose of at least about 0.1 mg to about 800 mg, about 1 to about 1000 mg, about 2 to about 1500 mg, about 5 to about 800 mg, about 5 to about 500 mg, or about 10 to about 400 mg. In certain embodiments, the initial dose may be followed by administration of a second or a plurality of subsequent doses of the PD-1 inhibitor in an amount that can be approximately the same or less than that of the initial dose, wherein the subsequent doses are separated by at least 1 day to 3 days; at least one week, at least 2 weeks; at least 3 weeks; at least 4 weeks; at least 5 weeks; at least 6 weeks; at least 7 weeks; at least 8 weeks; at least 9 weeks; at least 10 weeks; at least 12 weeks; or at least 14 weeks.

Various delivery systems are known and can be used to administer the pharmaceutical composition of the disclosure, e.g., encapsulation in liposomes, microparticles, microcapsules, recombinant cells capable of expressing the mutant viruses, receptor mediated endocytosis (see, e.g., Wu et al. (1987) J. Biol. Chem. 262:4429-4432). Methods of introduction include, but are not limited to, intradermal, transdermal, intramuscular, intraperitoneal, intravenous, subcutaneous, intranasal, epidural and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. The pharmaceutical composition can be also delivered in a vesicle, in particular a liposome (see, e.g., Langer (1990) Science 249:1527-1533).

The use of nanoparticles to deliver the PD-1 inhibitor of the present disclosure is also contemplated herein. Antibody-conjugated nanoparticles may be used both for therapeutic and diagnostic applications. Antibody-conjugated nanoparticles and methods of preparation and use are described in detail by Arruebo et al., 2009, “Antibody-conjugated nanoparticles for biomedical applications,” J. Nanomat., Vol. 2009, Article ID 439389, 24 pages. Nanoparticles may be developed and conjugated to antibodies contained in pharmaceutical compositions to target cells. Nanoparticles for drug delivery have also been described in, for example, U.S. Pat. Nos. 8,257,740, or 8,246,995.

In certain situations, the pharmaceutical composition can be delivered in a controlled release system. In one embodiment, a pump may be used. In another embodiment, polymeric materials can be used. In yet another embodiment, a controlled release system can be placed in proximity of the composition's target, thus requiring only a fraction of the systemic dose.

The injectable preparations may include dosage forms for intravenous, subcutaneous, intracranial, intraperitoneal and intramuscular injections, drip infusions, etc. These injectable preparations may be prepared by methods publicly known.

A pharmaceutical composition of the present disclosure can be delivered subcutaneously or intravenously with a standard needle and syringe. In addition, with respect to subcutaneous delivery, a pen delivery device readily has applications in delivering a pharmaceutical composition of the present disclosure. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the antibody contained is generally about 5 to about 1500 mg per dosage form in a unit dose; especially in the form of injection, it is preferred that the antibody is contained in about 5 to about 300 mg and in about 10 to about 300 mg for the other dosage forms.

In certain embodiments, the present disclosure provides a pharmaceutical composition or formulation comprising a therapeutic amount of a PD-1 inhibitor (e.g., an anti-PD-1 antibody or antigen-binding fragment thereof) and a pharmaceutically acceptable carrier. Non-limiting examples of pharmaceutical compositions comprising an anti-PD-1 antibody that can be used in the context of the present disclosure are disclosed in US 2019/0040137.

### Administration Regimens

In certain embodiments, the methods disclosed herein include administering to the tumor of a subject in need thereof a therapeutically effective amount of a PD-1 inhibitor (e.g., an anti-PD-1 antibody or antigen-binding fragment thereof) in multiple doses, e.g., as part of a specific therapeutic dosing regimen. For example, the therapeutic dosing regimen may comprise administering one or more doses of a PD-1 inhibitor to the subject at a frequency of about once a day, once every two days, once every three days, once every four days, once every five days, once every six days, once a week, once every two weeks, once every three weeks, once every four weeks, once every five weeks, once every six weeks, once every eight weeks, once every twelve weeks, once a month, once every two months, once every three months, once every four months, twice a day, twice every two days, twice every three days, twice every four days, twice every five days, twice every six days, twice a week, twice every two weeks, twice every three weeks, twice every four weeks, twice every five weeks, twice every six weeks, twice every eight weeks, twice every twelve weeks, twice a month, twice every two months, twice every three months, twice every four months, three times a day, three times every two days, three times every three days, three times every four days, three times every five days, three times every six days, three times a week, three times every two weeks, three times every three weeks, three times every four weeks, three times every five weeks, three times every six weeks, three times every eight weeks, three times every twelve weeks, three times a month, three times every two months, three times every three months, three times every four months or less frequently or as needed so long as a therapeutic response is achieved. In one embodiment, one or more doses of a PD-1 inhibitor are administered once every three weeks.

In certain embodiments, the one or more doses are administered in at least one treatment cycle. The methods, according to this aspect, comprise administering to a subject in need thereof at least one treatment cycle comprising administration of 1, 2, 3, 4, 5, 6, 7, 8, 9, 10 or more doses of a PD-1 inhibitor (e.g., an anti-PD-1 antibody or antigen-binding fragment thereof). In one embodiment, a treatment cycle comprises 12 doses of a PD-1 inhibitor. In one embodiment, a treatment cycle comprises 24 doses of a PD-1 inhibitor.

In certain embodiments, one or more doses of the PD-1 inhibitor are administered 1 to 12 weeks after the immediately preceding dose, for example, 1, 2, 3, 4, 5, 6, 7, 8, 9, 10, 11 or 12 weeks after the immediately preceding dose.

### Dosage

The amount of PD-1 inhibitor (e.g., an anti-PD-1 antibody or antigen-binding fragment thereof) administered to a subject according to the methods disclosed herein is, generally, a therapeutically effective amount. As used herein, the term “therapeutically effective amount” means an amount of a PD-1 inhibitor that results in one or more of: (a) a reduction in the severity or duration of a symptom or an indication of cancer—e.g., a tumor lesion; (b) inhibition of tumor growth, or an increase in tumor necrosis, tumor shrinkage and/or tumor disappearance; (c) delay in tumor growth and development; (d) inhibition of tumor metastasis; (e) prevention of recurrence of tumor growth; (f) increase in survival of a subject with a cancer; and/or (g) a reduction in the use or need for conventional anti-cancer therapy (e.g., elimination of need for surgery or reduced or eliminated use of chemotherapeutic or cytotoxic agents) as compared to an untreated subject or a subject treated with platinum based chemotherapy or other SOC therapy such as those disclosed herein.

In certain embodiments, a therapeutically effective amount of the PD-1 inhibitor (e.g., an anti-PD-1 antibody or antigen-binding fragment thereof) can be from about 0.05 mg to about 1500 mg, from about 1 mg to about 1050 mg, from about 1 mg to about 700 mg, from about 1 mg to about 600 mg, from about 10 mg to about 550 mg, from about 50 mg to about 400 mg, from about 75 mg to about 350 mg, or from about 100 mg to about 300 mg of the antibody. For example, in various embodiments, the amount of the PD-1 inhibitor is about 0.05 mg, about 0.1 mg, about 1.0 mg, about 1.5 mg, about 2.0 mg, about 5 mg, about 10 mg, about 15 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, about 900 mg, about 910 mg, about 920 mg, about 930 mg, about 940 mg, about 950 mg, about 960 mg, about 970 mg, about 980 mg, about 990 mg, about 1000 mg, about 1010 mg, about 1020 mg, about 1030 mg, about 1040 mg, about 1050 mg, about 1060 mg, about 1070 mg, about 1080 mg, about 1090 mg, about 1100 mg, about 1110 mg, about 1120 mg, about 1130 mg, about 1140 mg, about 1150 mg, about 1160 mg, about 1170 mg, about 1180 mg, about 1190 mg, about 1200 mg, about 1210 mg, about 1220 mg, about 1230 mg, about 1240 mg, about 1250 mg, about 1260 mg, about 1270 mg, about 1280 mg, about 1290 mg, about 1300 mg, about 1310 mg, about 1320 mg, about 1330 mg, about 1340 mg, about 1350 mg, about 1360 mg, about 1370 mg, about 1380 mg, about 1390 mg, about 1400 mg, about 1410 mg, about 1420 mg, about 1430 mg, about 1440 mg, about 1450 mg, about 1460 mg, about 1470 mg, about 1480 mg, or about 1500 mg.

The amount of a PD-1 inhibitor contained within an individual dose may be expressed in terms of milligrams of antibody per kilogram of subject body weight (i.e., mg/kg). In certain embodiments, the PD-1 inhibitor used in the methods disclosed herein may be administered to a subject at a dose of about 0.0001 to about 100 mg/kg of subject body weight. In certain embodiments, an anti-PD-1 antibody may be administered at dose of about 0.1 mg/kg to about 20 mg/kg of a patient's body weight. In certain embodiments, the methods of the present disclosure comprise administration of a PD-1 inhibitor (e.g., an anti-PD-1 antibody or antigen-binding fragment thereof) at a dose of about 1 mg/kg to 3 mg/kg, 1 mg/kg to 5 mg/kg, 1 mg/kg to 10 mg/kg, 1 mg/kg, 3 mg/kg, 5 mg/kg, or 10 mg/kg of a patient's body weight.

In certain embodiments, each dose comprises 0.1 - 10 mg/kg (e.g., 0.3 mg/kg, 1 mg/kg, 3 mg/kg, or 10 mg/kg) of the subject's body weight. In certain other embodiments, each dose comprises 5-1500 mg of the PD-1 inhibitor (such as an anti-PD-1 antibody or antigen-binding fragment thereof), e.g., 5, 10, 15, 20, 25, 40, 45, 50, 60, 70, 80, 90, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500 mg or more of the PD-1 inhibitor.

### EXAMPLES

The following examples are put forth so as to provide those of ordinary skill in the art with a complete disclosure and description of how to make and use the methods and compositions of the present disclosure and are not intended to limit the scope of what the inventors regard as their invention. Likewise, the disclosure is not limited to any particular preferred embodiments described herein. Indeed, modifications and variations of the embodiments may be apparent to those skilled in the art upon reading this specification and can be made without departing from its spirit and scope. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, room temperature is about 25° C., and pressure is at or near atmospheric.

### Example 1: Clinical Trial of anti-PD-1 Antibody in Patients with BCC after HHI Therapy

This study is a phase 2, non-randomized, 2-group, multi-center study of cemiplimab at a 350 mg dose administered intravenously (IV) every 3 weeks (Q3W) in patients with advanced BCC who experienced progression of disease on HHI therapy or were intolerant of prior HHI therapy. Cemiplimab is a high-affinity, human, hinge-stabilized IgG4 monoclonal antibody to the PD-1 receptor that potently blocks the interactions of PD-1 with PD-L1 and PD-L2. Cemiplimab comprises a heavy chain having the amino acid sequence of SEQ ID NO: 9 and a light chain having the amino acid sequence of SEQ ID NO: 10; an HCVR/LCVR amino acid sequence pair comprising SEQ ID NOs: 1/2; and heavy and light chain CDR sequences (HCDR1, HCDR2, HCDR3, LCDR1, LCDR2, LCDR3) comprising SEQ ID NOs: 3-8, respectively, as described herein. See also U.S. Pat. No. 9,987,500.

The study has 2 groups. Group 1 is for patients with metastatic BCC. Group 2 is for patients with unresectable locally advanced BCC. All patients underwent screening procedures to determine eligibility within 28 days prior to the initial administration of cemiplimab. There was no randomization or placebo control.

After a screening period of up to 28 days, patients received up to 93 weeks of treatment. Each patient received a 350 mg Q3W dose of cemiplimab IV. The infusion time for cemiplimab was approximately 30 minutes (±10 minutes). Tumor assessments were made at the end of each treatment cycle for 5 treatment cycles of 9 weeks followed by 4 treatment cycles of 12 weeks. Baseline assessments included digital medical photography and radiological imaging (CT or MRI) of all target lesions. Extensive safety evaluations occurred on day 1 of each cycle, with routine safety evaluations conducted at each cemiplimab dosing visit. Safety assessment was done continuously from initiation of study treatment until 105 days after the last study treatment. Patients were re-evaluated for response every 9 weeks in cycles 1-5, and every 12 weeks in cycles 6-9.

A patient received treatment until the 93-week treatment period was complete, or until disease progression (PD), unacceptable toxicity, withdrawal of consent, or confirmed complete response (CR). Patients with confirmed CR after a minimum of 48 weeks of treatment could elect to discontinue treatment and continue with all relevant study assessments (e.g., efficacy assessments). To establish a CR, biopsy of regressed target lesion documenting histological negativity was required.

### Study Objectives

A primary objective of the study was to estimate the objective response rate (ORR) for metastatic basal cell carcinoma (BCC) (Group 1) or unresectable locally advanced BCC (Group 2) when treated with cemiplimab monotherapy in patients who have progressed on Hedgehog Pathway Inhibitor (HHI) therapy, or were intolerant of prior HHI therapy

Secondary objectives of the study included: estimate ORR; estimate the duration of response, progression-free survival (PFS), and overall survival (OS); estimate the complete response (CR) rate; assess the safety and tolerability of cemiplimab; assess the pharmacokinetics (PK) of cemiplimab; assess the immunogenicity of cemiplimab; and assess the impact of cemiplimab on quality of life using European Organization for Research and Treatment of Cancer Quality of Life Questionnaire Core 30 (EORTC QLQ-C30) and Skindex-16.

Exploratory objectives of the study included: assess predictive potential and correlation to clinical response for biomarkers of interest including but not limited to tumor mutational burden.

### Rationale for Study Design

Basal cell carcinomas have a high mutational burden that encodes neoantigens for presentation to effector T (Teff) cells. Therefore, Teff cell responses against BCC will be unleashed by blockade of the PD-1 checkpoint with cemiplimab, achieving high ORR.

Several lines of evidence suggest that inhibition of the PD-1 checkpoint could be clinically advantageous for patients with advanced BCC. First, the mutational burden in BCC is among the highest of any human malignancy (Jayaraman et al., 2014, J Invest Dermatol, 134:213-220; Chalmers et al., 2016, AACR poster, abstract number 35762016; Bonilla et al., 2016, Nature Genetics, 48(4):398-406). Tumor types with high mutational burden are generally more responsive to PD-1 blockade than tumors with low mutational burden, and this is thought to be due to generation of neoantigens that can be recognized by Teff (Le et al., 2015, N Engl J Med, 372(26):2509-20; McGranahan et al., 2016, Science, 351:1463-69; Rizvi et al., 2015, Science, 345:124-28). Second, solid organ transplant patients have an approximately 10-fold increased risk of BCC, suggesting that immune surveillance is relevant in this disease (Euvrard et al., 2003, N Engl J Med, 348:1681-91). Third, other immune modulators have activity against BCC. The Toll-Like Receptor-7 (TLR-7) agonist imiquimod is an approved therapy for superficial BCC (Gollnick et al., 2008, Eur J Dermatol, 18(6):677-82). There is a case report of a BCC response to ipilimumab, an inhibitor of cytotoxic T-lymphocyte associated protein 4 (Mohan et al., 2016, JAAD Case Reports, 2:13-15). In a recent case report, disease stabilization of a previously progressing metastatic BCC was achieved with off-label administration of pembrolizumab (Winkler et al., 2016, Br J Dermatol, 176(2):498-502).

Because there is no standard of care for BCC patients who experienced progression of disease on HHI therapy, or are intolerant of prior HHI therapy, and metastatic and locally advanced disease are relatively rare, it has been acceptable to assess efficacy with non-randomized single-arm studies. Non-randomized studies without control arms, in which primary endpoints were ORR, were accepted by both the US Food and Drug Administration (FDA) and the European Medicines Agency (EMA) in the approvals of vismodegib and sonidegib for advanced BCC in the ERIVANCE (Migden 2015) and BOLT (Sekulic 2012) studies, respectively. Objective response rate is the primary endpoint in the study described herein.

Tumor biopsies are obtained at baseline and during treatment for patients with locally advanced tumors to inform an understanding of mechanisms of response and resistance to tumor treatment.

The decision to analyze separate groups of patients with metastatic (Group 1) and unresectable locally advanced (Group 2) BCC is based on the observation of higher response rates in locally advanced versus metastatic disease seen in data from studies of SMO inhibitors against BCC (Sekulic et al., 2012, N Engl J Med, 366:2171-9; Migden et al., 2015, Lancet Oncol, 16:716-28). This observation was also seen in a literature review of the reported experiences with other systemic therapies in CSCC, which demonstrates that response rates for various chemotherapy regimens generally are higher against advanced primary tumors that are locally advanced than against tumors that have metastasized to lymph nodes or distant visceral organs (Nakamura et al., 2013, Int J Clin Oncol, 18(3):506-09).

The rationale for including patients who are intolerant of HHIs is that such patients are unlikely to have a high probability of objective response if re-challenged with HHI. Because objective response tends to occur before onset of adverse events (AEs), it is unlikely that patients who interrupt HHI due to AEs will experience objective response upon re-challenge.

### Study Groups

Group 1: Patients with metastatic BCC. These patients are required to have histologic confirmation of distant BCC metastases (e.g., lung, liver, bone, or lymph node). Group 1 includes patients with both nodal metastatic and distant metastatic disease.

Group 2: Patients with unresectable locally advanced BCC. These patients are required to have disease that is considered inoperable, or to have medical contraindication to surgery or radiation, or have not achieved disease control with these treatments.

### Study Population

Patients with metastatic (Group 1) or unresectable locally advanced (Group 2) BCC who experienced progression of disease on HHI therapy, or were intolerant of prior HHI therapy.

Inclusion Criteria: A patient must meet the following criteria to be eligible for inclusion in the study: (1) histologically confirmed diagnosis of invasive BCC, including the following acceptable histologic subtypes of BCC: nodular, morpheaform, metatypical, superficial, micronodular, infiltrative, mixed, basosquamous, keratotic, desmoplastic; (2) patients must be deemed unlikely to benefit from further therapy with an HHI due to any of the following: (a) prior progression of disease on HHI therapy, or (b) intolerance of prior HHI therapy; (c) no better than a stable disease after 9 months on HHI therapy (exclusive of treatment breaks); (3) at least 1 lesion that is measurable by study criteria (Group 1: ≥mm in maximal diameter; Group 2: longest diameter and perpendicular diameter are both mm if measured by digital medical photography); (4) Eastern Cooperative Oncology Group (ECOG) performance status ≤1; (5) at least 18 years old; (6) hepatic function: (a) total bilirubin ≤1.5×upper limit of normal (ULN) (or ≤3×ULN, if liver metastases); (b) transaminases ≤3×ULN (or ≤5×ULN, if liver metastases); (c) alkaline phosphatase (ALP) ≤2.5×ULN (or ≤5×ULN, if liver or bone metastases); (7) renal function: werum creatinine ≤2×ULN or estimated creatinine clearance >35 mL/min; (8) creatine phosphokinase (CPK) elevation ≤grade 2; (9) bone marrow function: (a) hemoglobin ≥9.0 g/dL; (b) absolute neutrophil count (ANC)×109/L; (c) platelet count ≥75×109/L; (10) anticipated life expectancy >12 weeks; (11) consent to provide archived or newly obtained tumor material for central pathology review for confirmation of diagnosis of BCC; (12) Group 2 only (unresectable locally advanced BCC): patients must consent to undergo biopsies of externally visible BCC lesions at baseline, cycle 1 day 22 (±3 business days), at time of tumor progression, and at other time points that may be clinically indicated; (13) willing and able to comply with clinic visits and study-related procedures; (14) informed consent prior to any screening procedures; (15) Group 2 only: patients must be deemed to have unresectable disease. Surgery must be deemed contraindicated in the opinion of a Mohs dermatologic surgeon, a head and neck surgeon, or plastic surgeon. Acceptable contraindications include: (a) BCC that has recurred in the same location after 2 or more surgical procedures and curative resection is deemed unlikely; (b) BCCs with significant local invasion that precludes complete resection; (c) BCCs in anatomically challenging locations for which surgery may result in severe disfigurement or dysfunction (e.g., removal of all or part of a facial structure, such as nose, ear, or eye; or requirement for limb amputation); (16) Group 2 only: patients must be deemed as not appropriate for radiation therapy and must meet at least 1 of the following criteria: (a) previously received radiation therapy for BCC, such that further radiation therapy would exceed the threshold of acceptable cumulative dose, per the radiation oncologist; (b) tumor is unlikely to respond to therapy; (c) radiation therapy is deemed to be contraindicated; acceptable contraindications to radiation therapy for patients who have not received any prior radiation include: BCCs in anatomically challenging locations for which radiation therapy would be associated with unacceptable toxicity risk).

Exclusion Criteria: A patient who meets any of the following criteria is excluded from the study: (1) ongoing or recent (within 5 years) evidence of significant autoimmune disease that required treatment with systemic immunosuppressive treatments, which may suggest risk for immune-related adverse events (irAEs), except for: vitiligo, childhood asthma that has resolved, type 1 diabetes, residual hypothyroidism that required only hormone replacement, or psoriasis that does not require systemic treatment; (2) prior treatment with an agent that blocks the PD-1/PD-L1 pathway; (3) prior treatment with other systemic immune-modulating agents within fewer than 28 days prior to the first dose of cemiplimab (e.g., therapeutic vaccines, cytokine treatments, or agents that target cytotoxic T lymphocyte antigen 4 (CTLA-4), 4-1BB (CD137), or OX-40); (4) untreated brain metastasis(es) that may be considered active; (5) immunosuppressive corticosteroid doses (>10 mg prednisone daily or equivalent) within 4 weeks prior to the first dose of cemiplimab; (6) active infection requiring therapy, including positive tests for human immunodeficiency virus (HIV)-1 or HIV-2 serum antibody, hepatitis B virus (HBV), or hepatitis C virus (HCV); (7) history of pneumonitis within the last 5 years; (8) any anticancer treatment other than radiation therapy (chemotherapy, targeted systemic therapy, imiquimod, photodynamic therapy), investigational or standard of care, within 30 days of the initial administration of cemiplimab or planned to occur during the study period (patients receiving bisphosphonates or denosumab allowed); (9) history of documented allergic reactions or acute hypersensitivity reaction attributed to antibody treatments; (10) patients with allergy or hypersensitivity to cemiplimab or to any of the excipients; (11) concurrent malignancy other than BCC and/or history of malignancy other than BCC within 3 years of date of first planned dose of cemiplimab, except for tumors with negligible risk of metastasis or death, such as adequately treated CSCC of the skin, carcinoma in situ of the cervix, or ductal carcinoma in situ of the breast, or low-risk early stage prostate adenocarcinoma (T1-T2a NOMO and Gleason score <6 and PSA <10 ng/mL) for which the management plan is active surveillance, or prostate adenocarcinoma with biochemical-only recurrence with documented PSA doubling time of >12 months for which the management plan is active surveillance (D'Amico 2005; Pham 2016); (12) any acute or chronic psychiatric problems that make the patient ineligible for participation; (13) patients with a history of solid organ transplant; (14) any medical co-morbidity, physical examination finding, or metabolic dysfunction, or clinical laboratory abnormality that renders the patient unsuitable for participation; (15) inability to undergo any contrast-enhanced radiologic response assessment; (16) breastfeeding; (17) positive serum pregnancy test; (18) receipt of live vaccines (including attenuated) within 30 days of first study treatment; (19) women of childbearing potential (WOCBP), or sexually active men, who are unwilling to practice highly effective contraception prior to the initial dose/start of the first treatment, during the study, and for at least 6 months after the last dose; (20) prior treatment with idelalisib.

### Study Treatment

Open-label cemiplimab was supplied as a liquid in sterile, single-use vials. Each vial contained cemiplimab at a concentration of 50 mg/mL. Cemiplimab was administered in an outpatient setting as an IV infusion over 30-minutes (±10 minutes). Each patient's dose was administered as a flat dose of 350 mg Q3W. No premedications were administered for the first dose of cemiplimab.

### Concomitant Medications and Procedures

While participating in the study, a patient may not receive any standard or investigational agent for treatment of a tumor other than cemiplimab as monotherapy. For patients with locally advanced target lesions that are considered unresectable at baseline, but are subsequently deemed resectable during the course of the study due to tumor response to cemiplimab, curative intent surgery may be allowed. Patients with inoperable BCC at baseline who are rendered operable with clear margins are deemed to have experienced PR. Radiation therapy is not part of the study regimen.

### Study Endpoints

The primary efficacy endpoint for this study is the ORR, which was assessed separately for patients with metastatic BCC (Group 1) or unresectable locally advanced BCC (Group 2). For patients in Group 1 (metastatic BCC), ORR was determined by Response Evaluation Criteria in Solid Tumors (RECIST) version 1.1 for visceral lesions or by modified WHO criteria for skin lesions, or by composite response criteria for patients with both visceral and skin lesions. Clinical response criteria may be used for patients with externally visible target lesions if all metastatic lesions are not measurable by RECIST (as may occur in patients with bone-only metastases). For patients in Group 2 (unresectable locally advanced BCC), clinical criteria were used to determine ORR. Composite response criteria were used for patients with lesions that were measurable by both clinical response criteria and RECIST 1.1.

Secondary endpoints are: objective response; duration of response, defined as the time between first measurement of complete or partial response and the first date of recurrent or progressive disease or death; PFS, defined as the time between start of treatment and the first date of recurrent or progressive disease or death from any cause; OS, defined as the time between the start of treatment and death from any cause; CR rate; change in scores of patient-reported outcomes in the EORTC QLQ-C30 and the Skindex-16; adverse events (AEs); concentrations of cemiplimab in serum; anti-cemiplimab antibodies; proportion of patients attaining best response of CR; time to response, defined as the time between start of treatment and the first best response of complete or partial response (whichever comes first); and safety and tolerability of cemiplimab. The secondary efficacy endpoints, DOR, PFS, and OS, were estimated using the Kaplan-Meier (KM) method.

Additional secondary outcome measures included disease control, defined as the proportion of patients with a best response of complete response, partial response, stable disease, or non-partial response or non-progressive disease at the first evaluable tumor assessment, scheduled to occur at week 9 (defined as 56 days to account for visit windows in the protocol); and durable disease control, defined as the proportion of patients without progressive disease for at least 182 days.

The following exploratory analyses were planned: associations between tumor non-synonymous mutational burden at baseline and efficacy of cemiplimab; pharmacodynamic changes, comparing baseline and on-treatment biopsies: changes in tumor mRNA expression; changes in number of TI Ls (CD8+ T cells, CD4+ T cells, T regulatory cells, and tissue permitting, other subtypes such as B cells, myeloid-derived cells, NK cells, etc.) and descriptive change in distribution of TILs in respect to tumor tissue and stroma; change in expression levels (mRNA and/or protein) of PD-L1, GITR, and LAG-3, and possibly other check-point modulators; and change in number and type of genetic mutations in known oncogenes and potential tumor neoantigens.

### Response Criteria

Complete Response (CR): Disappearance of all target lesions. Any pathological lymph nodes (whether target or non-target) must have reduction in short axis to <10 mm (<1 cm).

Partial Response (PR): At least a 30% decrease in the sum of the diameters of target lesions, taking as reference the baseline sum diameters.

Progressive Disease (PD): At least a 20% increase in the sum of the diameters of target lesions, taking as reference the smallest sum on study (this includes the baseline sum if that is the smallest on study). In addition to the relative increase of 20%, the sum must also demonstrate an absolute increase of at least 5 mm (0.5 cm).

Stable Disease (SD): Neither sufficient shrinkage to qualify for PR nor sufficient increase to qualify for PD, taking as reference the smallest sum diameters while on study.

### Procedures and Assessments

Tumor imaging (computed tomography [CT] or magnetic resonance imaging [MRI]) and digital medical photography (for externally visible lesions) were performed to measure tumor burden and to characterize the efficacy profile of study treatments using response criteria. Physical examination, laboratory tests, vital signs, electrocardiogram (ECG), pregnancy test for women of childbearing potential, and recording of AEs and concomitant medications was performed to ensure patient safety and to characterize the safety profiles of study treatments. Other assessments included: Blood samples for PK; Blood samples to assess anti-cemiplimab antibodies; Tumor biopsies; Biomarkers; Quality of life assessments.

Baseline assessments included digital medical photography and radiologic imaging (computed tomography [CT] or magnetic resonance imaging) of all target lesions. CT chest was required during the screening period to rule out metastatic disease. For tumor assessments at the end of each treatment cycle, repeat of the same photographic and radiologic assessments completed at baseline were encouraged. However, in cases where baseline imaging (photography and radiology) showed that the disease was comprehensively assessed by one modality (photography or radiology), post-baseline assessments could be only photography (or radiology). To establish CR, biopsy of regressed target lesion documenting histologic negativity was required. All responses were required to be confirmed by two separate tumor assessments, at least 4 weeks apart. If the last tumor assessment prior to the data cut-off was the first documentation of response, the centrally reviewed tumor assessment subsequent to data cut-off was allowed to confirm response status. Adverse events and laboratory abnormalities were graded according to the National Cancer Institute Common Terminology Criteria for Adverse Events, version 4.03.

ORR was defined as CR+partial response (PR). After any objective response, confirmatory digital photography (and radiologic imaging, if performed as part of the initial response assessment) was obtained at least 4 weeks following initial documentation of objective response.

Pre-treatment tumors were used to explore potential biomarkers including expression of selected proteins (PD-L1, major histocompatibility complex class-I [MHC-I]) by immunohistochemistry (IHC), and tumor mutation burden (TMB). To explore potential mechanisms of immune evasion associations between percent of tumor cells positive for MHC-I expression and objective response, assessments were made in tumors with high and low TMB (≥10 and <10 mut/Mb, respectively). MHC-I expression scoring was based on quantitative image analysis, and the MHC-I positive percentage was calculated as the number of MHC-I positive tumor cells divided by the total number of tumor cells, multiplied by 100.

### PD-L1 Expression and TMB

PD-L1 expression level was assessed by the PD-L1 immunohistochemistry (IHC) 22C3 assay (Dako, Agilent, Santa Clara, CA) in formalin-fixed paraffin embedded (FFPE) biopsy samples obtained prior to cemiplimab therapy. Expression level was quantified as the percentage of tumor cells with detectable PD-L1 membrane staining (tumor proportion score [TPS]). Tumor mutational burden (TMB) was estimated in the DNA samples extracted from the FFPE tumor biopsies using the analytically validated TruSight Oncology 500 (IIlumina Inc., San Diego, CA) to detect single nucleotide variants (SNV), insertions and deletions (indels), and copy number alterations (CNV) in 500 genes and selected sets of gene rearrangements. TMB was calculated as the total number of somatic SNVs and indels in the coding regions of targeted genes per megabase of analyzed genomic sequence. All somatic mutations were filtered to exclude the germline and oncogenic driver gene variants, according to the public database comparisons. The assay protocol included the addition of unique molecular identifier (UMI) nucleotide barcodes during the sequencing library generation. The detection of UMIs is used to identify sequence reads from complementary DNA strands, in order to reduce the effect of FFPE DNA deamination artefacts on mutational variant calling.

### Multiplex IHC Assay

A fully automated multiplex IHC assay was performed on the Ventana Discovery ULTRA platform (Ventana Medical Systems, Tucson, AZ), and as previously described (Zhang et al., 2017, Laboratory Investigation, 873-885). Five rounds of sequential primary antibody and secondary-horseradish peroxidase-conjugated antibody applications were performed. Heat denaturation between each step to completely remove the bound primary and secondary antibody was performed to eliminate downstream cross-reactivity. This allowed primary antibodies raised in the same species to be used.

The fluorescent dyes used were carefully selected to ensure spectral separation and provide optimal staining. The combination and order of application of the primary antibody and tyramide-fluorophore was optimized to ensure that both the epitope and fluorophore could withstand the repeated heat denaturation steps.

The assay was optimized for the specific tumor indication. The optimal concentrations of each antibody were determined, and they were applied in the following sequence and detected with the indicated fluorophore: (1) Mouse anti-MHCII (ABCAM, Clone CR3/43) was detected with DISCOVERY Rhodamine 6G. Mouse anti-PAN CK (Ventana, Clone AE1/AE3/PCK26) was detected with DISCOVERY DCC; (2) Rabbit anti-CD11c (ABACM, Clone EP1347Y) was detected with DISCOVERY Rhodamine 610; (3) Rabbit anti-MHCI (ABCAM, Clone SP239) was detected with DISCOVERY Cy5; (4) Rabbit anti-B2M (ABCAM, Clone EPR217520214) was detected with DISCOVERY FAM.

Following staining, the tissue was counter-stained and cover-slipped with Invitrogen ProLong Gold Antifade Mountant with NucBlue. Whole-slide imaging was performed on the Zeiss Axioscan which was equipped with a Colibri light source and appropriate filters for visualizing these specific fluorophores. Image analysis was performed using HALO Indica Labs software modules (Indica Labs, Albuquerque, NM).

The fraction of MHC-positive cells in each tumor region was scored by the HALO image analysis software. Tumor regions were demarcated, and individual cells identified by Dapi staining. The total number of tumor cells was determined by examining the cells positive for Dapi and panCK. The fraction of MHC-I positive tumor cells was then calculated as the percentage of Dapi-panCK and MHC-I positive cells over the total number of tumor cells (Dapi and panCK-positive).

### Results (Group 2): Locally Advanced BCC (laBCC) Patients

Patient Characteristics: The results set forth herein are based on 84 patients with laBCC who were enrolled; 56 (66%) were male; median age was 70 years (range, 42 to 89). The primary tumor site was the head and neck in 75 (89%); trunk in seven (8%) and extremities in two (2%) patients. See Table 1. At the time of data cut-off, 19 patients remained on treatment, 13 patients had completed planned treatment (93 weeks) and 52 patients had discontinued, mainly due to disease progression (n=29). The median number of administered doses was 15 (range, 1 to 31). Median duration of exposure was 47 weeks (range 2 to 94). Median duration of follow-up was 15 months (range, 0.5 to 25). The enrolled laBCC patients had progressed on or were intolerant to previous HHI therapy. Patients were not candidates for further HHI therapy due to progression of disease on or intolerance to previous HHI therapy or having no better than stable disease after 9 months on HHI therapy; and had at least one baseline lesion measurable by digital medical photography per modified WHO criteria or by radiological imaging (CT or MRI) as per RECIST 1.1 criteria. Patients were not candidates for curative surgery or curative radiotherapy.

TABLE 1
Baseline Patient Characteristics and Exposure to Cemiplimab

| | Locally | |
|---|---|---|
| | Advanced BCC | |
| Characteristics, n (%), Unless Otherwise Stated | (n = 84) | |
| Median age, years (range) | 70 | (61-79) |
| ≥65 years | 53 | (63%) |
| Sex | | |
| Male | 56 | (67%) |
| Female | 28 | (33%) |
| ECOG PS score | | |
| 0 | 51 | (61%) |
| 1 | 33 | (39%) |
| Number of patients with prior cancer-related radiotherapy | 42 | (50%) |
| Number of patients with prior HHI therapy | | |
| Vismodegib | 79 | (94%) |
| Sonidegib | 14 | (17%) |
| Vismodegib + Sonidegib | 9 | (11%) |
| Reason for discontinuation of prior HHI* | | |
| Progression of disease on HHI | 60 | (71%) |
| Intolerant to prior HHI therapy | 32 | (38%) |
| Intolerant to vismodegib | 32 | (38%) |
| Intolerant to sonidegib | 4 | (5%) |
| No better than stable disease after 9 months | 7 | (8%) |
| on HHI therapy | | |
| Primary BCC site | | |
| Head and neck | 75 | (89%) |
| Trunk | 7 | (8%) |
| Extremity (arm or leg) | 2 | (2%) |
| Median (range) duration of exposure, weeks | 47 | (2-94) |
| Median (range) number of doses administered | 15 | (1-31) |

Data are median (IQR) or n (%).
*Sum is >84 because some patients had more than one reason for discontinuation.

Clinical Efficacy: As summarized in Table 2, the ORR was 31% (95% Confidence Interval (CI), 21 to 42), including five (6%) CRs. The median time to response was 4.3 months (range, 4.2 to 7.2). The disease control rate was observed in 67 of 84 patients, 80% (95% CI, 70 to 88); and the durable disease control rate was 60% (95% CI, 48 to 70), observed in 50 patients. Median DOR had not been reached at the time of data cut-off. KM estimates for DOR at 6 and 12 months were 91% (95% CI, 68 to 98) and 85% (95% CI, 61 to 95), respectively.

TABLE 2
Tumor Response and Duration of Response

| | |
|---|---|
| | Locally |
| | Advanced BCC |
| n (%), Unless Otherwise Stated | (n = 84) |

Best overall response

| Overall response rate, % (95% CI) | 26 | (31%; 21-42)^{a} |
|---|---|---|
| Complete response | 5 | (6%) |
| Partial response | 21 | (25%) |
| Stable disease | 41 | (49%) |
| Progressive disease | 9 | (11%) |
| Not evaluable ^{b} | 8 | (10%) |
| Disease control rate, % (95% CI) ^{c} | 67 | (80%; 70-88) |
| Durable disease control rate, % (95% CI) | 50 | (60%; 48-70) |
| Median (range) time to response, months ^{d} | 4.3 | (4.2-7.2) |
| Observed duration of response, range, months ^{d} | 2-21 | |
| ≥6 months | 19 | (79%) |
| ≥12 months | 11 | (46%) |
| Kaplan-Meier estimation of duration of response, | Not reached (15- | |
| median (95% CI), months ^{d} | not evaluable) | |
| 6 months | 91% | (68-98) |
| 12 months | 85% | (61-95) |
| Probability of progression-free survival, | | |
| % (95% CI) | | |
| 6 months | 76 | (65-84) |
| 12 months | 57 | (44-67) |

Data are n (%; 95% CI), n (%), median (IQR), or range (where specified).
*Objective response per independent central review includes two partial responses that emerged at tumor assessments before the data cutoff and were confirmed by tumor assessments done subsequent to the data cutoff.
^{a}ORR includes two partial responses that emerged at tumor assessments prior to data cut-off, and were confirmed by tumor assessments performed subsequent to the data cut-off. ORR was observed in 27 or 84 patients, 32% (95% CI, 22 to 43), including five (6%) complete responses and 22 (26%) partial responses.
^{b} Of the eight patients who were not evaluable, four did not have any post-baseline tumor assessments. Three patients were not considered to have evaluable lesions by either photographic or radiologic assessment methods. One patient had a second target lesion not imaged after baseline.
^{c} Defined as the proportion of patients with CR, PR, SD or Non-PR/Non-PD at the first evaluable tumor assessment, scheduled to occur at week 9 (defined as 56 days to account for visit windows in the protocol)
^{d} Data shown are for patients with a confirmed complete response or partial response; duration of response was calculated for all patients with a confirmed response prior to the data cutoff.

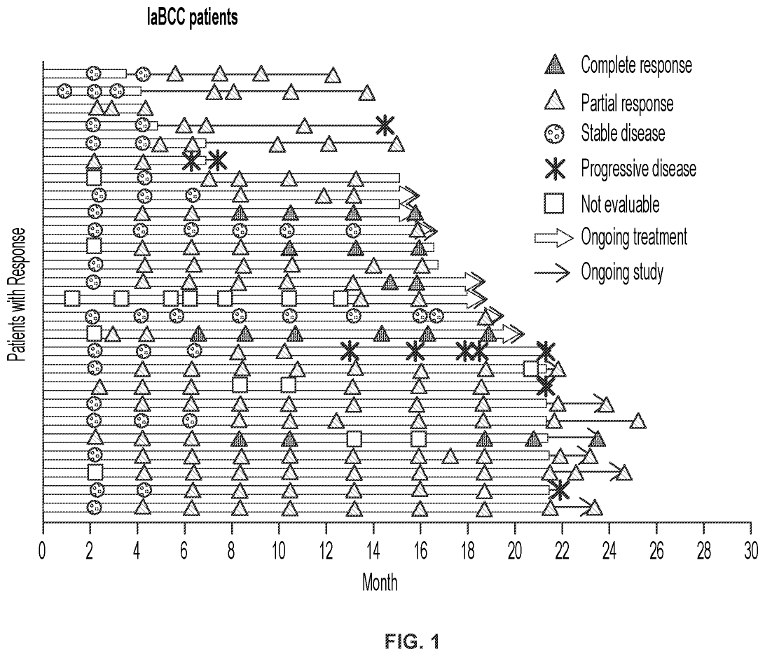

FIG. 1 provides swimmer plots that depict both time to response, and duration of response of 26 patients with locally advanced BCC. In this figure, the closed arrow indicates the patient was still on treatment; and the open arrow indicates the patient was still on study. Each horizontal bar represents one patient. Among the 26 patients with confirmed responses at data cut-off, only five had evidence of subsequent disease progression. Many of the responses deepen over time. Median Kaplan-Meier (KM) estimation of progression-free survival (PFS) was 19 months (95% Confidence Interval (CI), 9 to not evaluable). The KM estimated 12-month probability of PFS was 57% (95% CI, 44 to 67); and the KM estimated 6-month probability of PFS was 76% (95% CI, 65 to 84). In subgroup analyses, efficacy was similar regardless of baseline characteristics, including reason for discontinuation of prior HHI therapy.

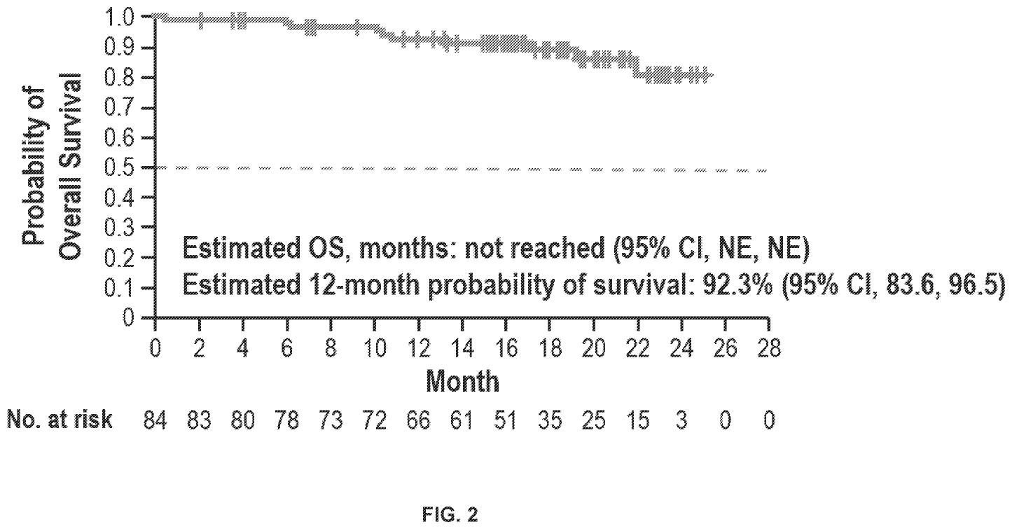

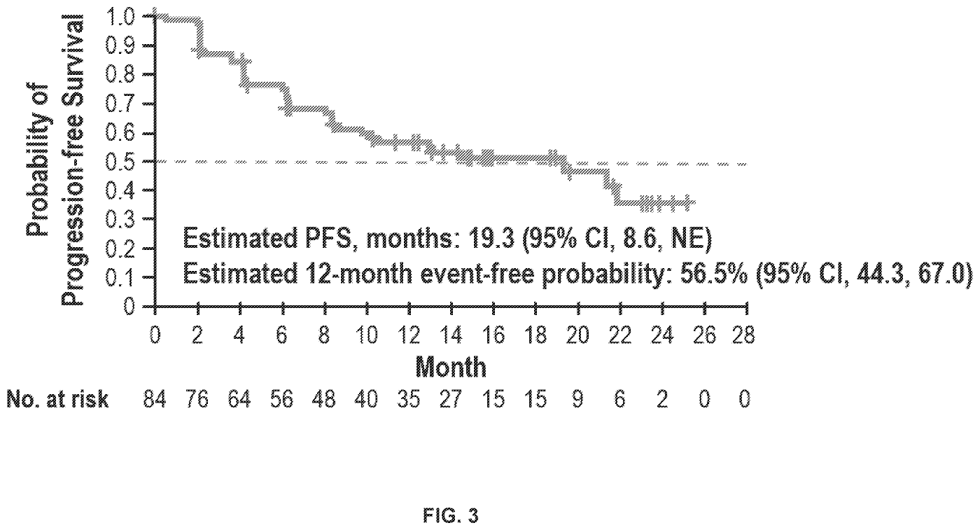

FIG. 2 shows estimated OS (months) was not reached (95% CI, NE, NE); and estimated 12-month probability of survival was 92.3% (95% CI, 83.6, 96.5). FIG. 3 shows estimated PFS (months) was 19.3 (95%, 8.6, NE). Estimated 6-month PFS was 76% (95% CI, 65-84), and estimated 12-month PFS was 56.5% (95% CI, 44.3, 67.0).

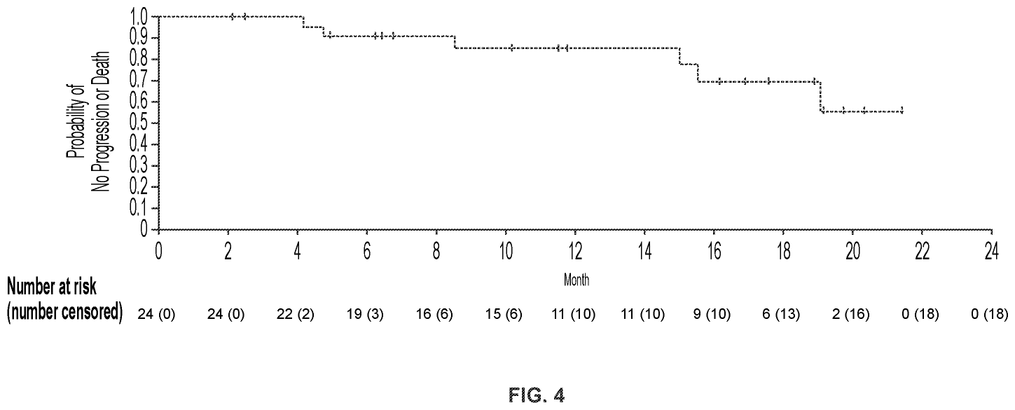

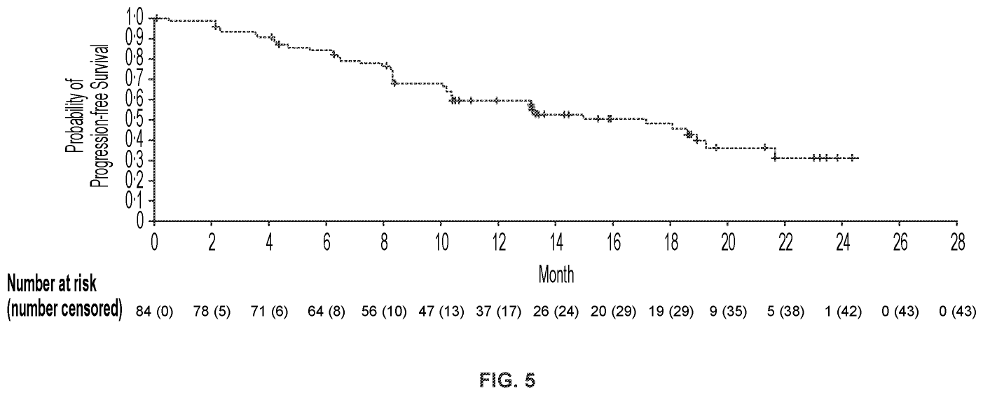

FIG. 4 shows KM estimates for duration of response at 6 and 12 months were 91% (95% CI 68-98) and 85% (95% CI 61-95), respectively. FIG. 5 shows KM estimate of median PFS was 17 months (95% CI 10-19); probability of PFS at 6 months was 85% (95% CI 74-91); and probability of PFS at 12 months was 59% (95% CI 47-70).

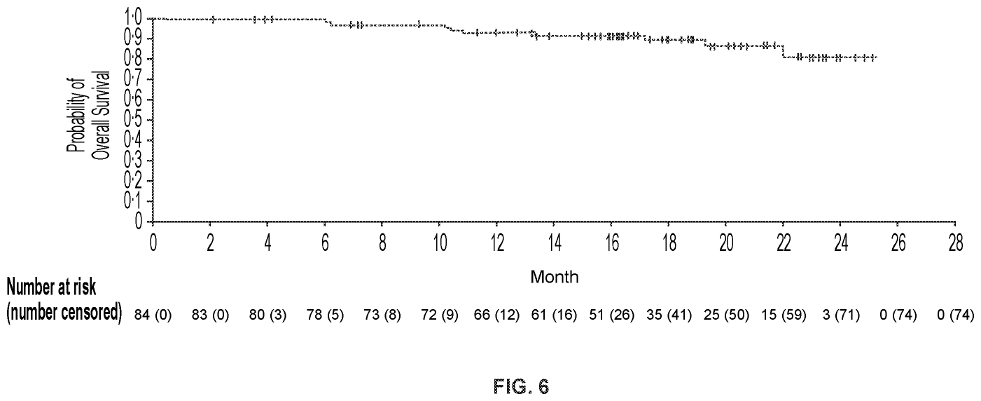

FIG. 6 shows KM estimates of OS, wherein median OS had not been reached at the time of data cutoff. KM estimated proportion of patients alive at 2 years was 80% (95% CI, 63-90).

Table 3 shows that, in subgroup analyses, clinical activity was similar regardless of baseline characteristics.

TABLE 3
Subgroup Analysis of Response

| Subgroup | Responder, n (%) |
|---|---|
| Overall population: (N = 84) | 26 (31) |
| Sex: male (n = 56) | 17 (30) |
| Sex: female (n = 28) | 9 (32) |
| Age group: <65 (n = 31) | 10 (32) |
| Age group: ≥65 (n = 53) | 16 (30) |
| Outcome of prior HHI therapy: disease progression/ | 18 (29) |
| lack of response (n = 63) | |
| Outcome of prior HHI therapy: intolerance (n = 21) | 8 (38) |

Biomarkers: Baseline tumor samples were evaluable for PD-L1 IHC in 50 (60%) of 84, for TMB in 56 (66%) of 84 patients, and for MHC-I IHC in 44 (52%) of 84 patients. Among some patients with high TMB who did not have objective responses, MHC-I expression level on tumor cells was low or absent. ORR was 26% (95% CI, 13 to 43) in 35 patients with PD-L1<1% and 27% (95% CI, 8 to 55) in 15 patients with PD-L1≥1%, as summarized in Table 4. Objective responses were observed in patients regardless of baseline PD-L1 levels.

TABLE 4
Best Overall Tumor Response Rate by Positive PD-L1

| | Evaluable PD-L1 (n = 50) | | | |
|---|---|---|---|---|
| | PD-L1 <1% | | PD-L1 ≥1% | |
| n (%) | (n = 35) | | (n = 15) | |
| Overall response rate, % (95% CI) | 26 (13-43) (26) | | 27 | (8-55) |
| Complete response | 2 | (6) | 2 | (13) |
| Partial response | 7 | (20) | 2 | (13) |
| Stable disease | 18 | (51) | 9 | (60) |
| Non-complete response/non-progressive | 0 | | 0 | |
| disease | | | | |
| Progressive disease | 5 | (14) | 1 | (7) |
| Not evaluable | 3 | (9) | 1 | (7) |
| Disease control rate, % (95% CI)* | 77 | (60-90) | 87 | (60-98) |
| Durable disease control rate, % (95% CI) ^{†} | 51 | (34-69) | 53 | (27-79) |

*Defined as the proportion of patients with complete response, partial response, stable disease or non-partial response/non-progressive disease at the first evaluable tumor assessment, scheduled to occur at week 9.
^{†} Defined as the proportion of patients with complete response, partial response, stable disease or non-partial response/non-progressive disease for at least 27 weeks without progressive disease (defined as 182 days).

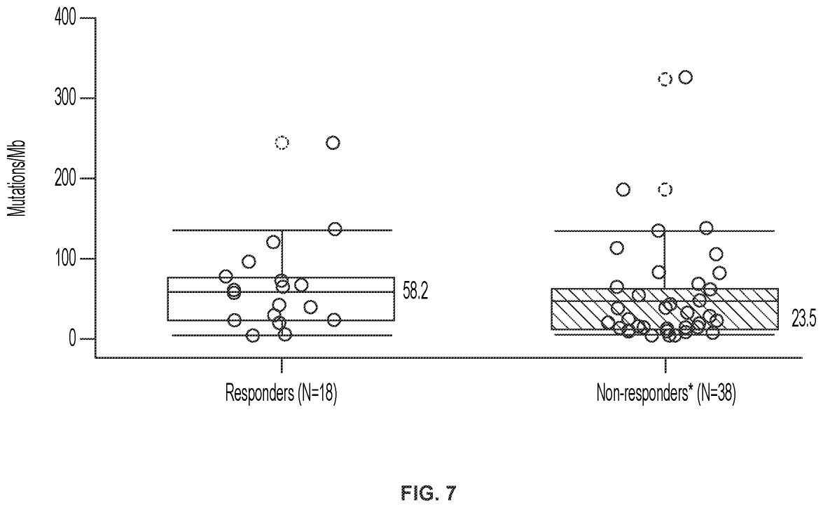

Median TMB was 58.2 mut/Mb and 23.5 mut/Mb among responding (PR or CR) and non-responding patients, respectively, as shown in FIG. 7. This figure depicts TMB for responders (complete or partial response) versus non-responders (stable disease, progressive disease, or not evaluable). Lines in each box denote median; lower and upper boundaries of box denote lower quartile and upper quartile (IQR), respectively; and upper and lower whiskers indicate maximum (Q3+1.5*IQR) and minimum (Q1−1.5*IQR) values, respectively. Individual patients are indicated by open circles. Open circles beyond the whiskers are outliers.

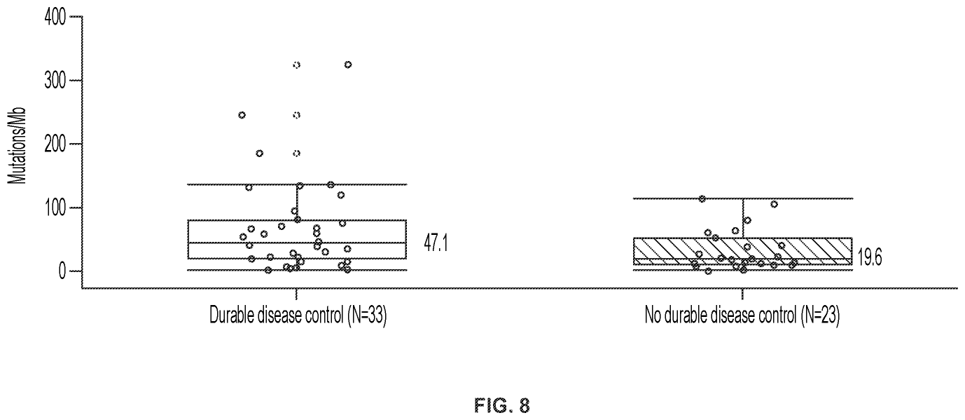

Not all patients with high TMB tumors had responses to treatment, and some patients with low TMB tumors had responses to treatment, as shown in FIG. 8. This figure depicts TMB for patients who achieved durable disease control (patients without progressive disease for at least 182 days) versus those who did not. Lines in each box denote median; lower and upper boundaries of box denote lower quartile and upper quartile (IQR), respectively; and upper and lower whiskers indicate maximum (Q3+1.5*IQR) and minimum (Q1−1.5*IQR) values, respectively. Individual patients are indicated by open circles. Open circles beyond the whiskers are outliers.

When using the 10 mut/Mb cutoff, 21 individuals (9 responders, 12 non-responders) were in the high TMB group and had evaluable sample for MHC-I testing. In this high TMB group, the median proportions of tumor cells positive for MHC-I expression were 39% (Q1−Q3, 23 to 48%) and 5% (Q1−Q3, 3 to 12%) in responders and non-responders, respectively. In the low TMB group (<10 mut/Mb), the proportions of tumor cells positive for MHC-I were 77% in one responder and median 47%.

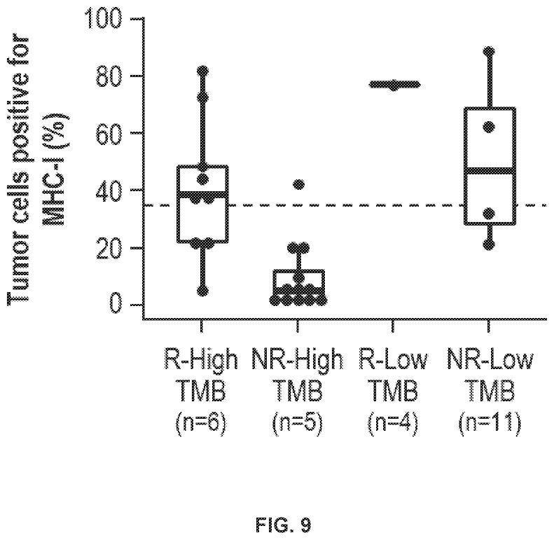

Q1−Q3, 29 to 69%) among four non-responders. These results are shown in FIG. 9, which depicts 21 patients (9 responders and 12 non-responders) in the high TMB group (≥10 mut/Mb), and 5 patients (one responder and four non-responders) in the low TMB group (<10 mut/Mb). The horizontal broken line indicates the threshold for a clinically meaningful change. In the high TMB group (10 mutations/MB), responders exhibited median 38.6% MHC-I (+) tumor cells; and non-responders exhibited median 5.1% MHC-I (+) tumor cells (FIG. 6).

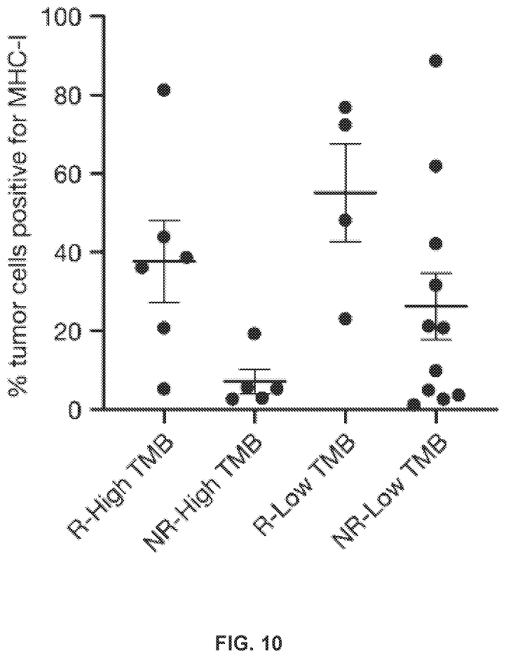

The association between MHC-I expression and ORR is also observed if high TMB is defined as above the overall median of 34.6 mut/Mb, as shown in FIG. 10. In this figure, median TMB was higher in responders vs. non-responders in the high TMB group (>10 mut/Mb; FIG. 9). This general trend was preserved when high TMB was defined as above the median of 34.6 mut/Mb. Representative examples of positive and negative MHC-I staining in pretreatment samples from study patients were also obtained, including a patient with complete response (TMB: 67.398 mutations/Mb) and a patient with progressive disease (TMB: 81.432 mutations/Mb).

Adverse Events: The most common AEs, of any grade, regardless of attribution were fatigue (30%), diarrhea (24%), pruritus (21%), and asthenia (20%). Grade AEs occurred in 51% of patients. The most common grade AEs occurring in patients were hypertension (n=4; 5%) and fatigue, urinary tract infection, and visual impairment (each n=3; 4%). Fourteen patients (17%) discontinued treatment due to AEs.

The most common treatment-related AEs (TRAEs) included fatigue (n=21; 25%), pruritus (n=12; 14%), and asthenia (n=12; 14%). Grade TRAEs occurred in 20% of patients. The most common grade TRAEs were fatigue, colitis, autoimmune colitis and adrenal insufficiency (n=2 each). Nine patients (11%) discontinued treatment due to TRAEs.

There were no treatment-related deaths. Three deaths, due to treatment-emergent adverse events considered to be related to intercurrent medical issues were reported. They included a 55-year-old woman with new intracranial sarcoma arising from transformation of known meningioma; an 85-year-old man with acute-on-chronic renal failure in the setting of suspected septic pneumonia; and a 73-year-old man with history of malnutrition, who died due to cachexia.

Identified irAEs occurred in 21 (25%) patients. The most common were hypothyroidism and immune-related colitis, occurring in 8 (10%) and 5 (6%) patients, respectively. irAEs were grade 3 in 10% (n=8) of patients. The following grade 3 irAEs occurred in >1 patient: immune-related colitis (n=3), adrenal insufficiency (n=2). There were no grade 4 or grade 5 irAEs.

### Discussion

Studies of immune checkpoint blockade in melanoma were followed by demonstrations that PD-1/PD-L1 blockade is a highly active therapy against advanced CSCC and Merkel cell carcinoma (Barrios et al., J Am Acad Dermatol, May 2020). For patients with laBCC, there is no effective therapy after first-line HHI therapy. The pivotal study described above shows clinically meaningful anti-tumor activity in patients with laBCC in the second-line (or greater) setting. Centrally reviewed ORR is 31% (95% CI, 21 to 42%). Estimated DOR exceeded 1 year in 85% of responders. The safety profile is consistent with what is known for the anti-PD-1/PD-L1 class.

The results of the present study fill a long-standing gap regarding lack of treatment options for laBCC patients after first-line HHI therapy. Objective responses with HHI therapy occur in approximately half of patients with laBCC, but most do not achieve CRs (Dummer et al., 2020, Br J Dermatol, 182:1369-78; Sekulic et al., 2015, J Am Acad Dermatol, 72:1021-26 e8). Among those patients who respond to HHI therapy, the median duration of response was 26 months, underscoring the development of resistance to HHI therapy leading to loss of response (Sekulic et al., 2017, BMC Cancer, 17:332; Dummer et al., 2020, Br J Dermatol, 182:1369-78). Toxicities of the HHI class include dysgeusia, muscle spasms, and alopecia. Although toxicity was the most common reason for treatment discontinuation in the largest prospective studies of vismodegib (Basset-Seguin et al., 2017, Eur J Cancer, 86:334-48; Dréno et al., 2017, Lancet Oncol, 18:404-12), the most common reason for discontinuation of prior HHI therapy in the current study was disease progression. Therefore, the patient population enrolled in this cemiplimab study represents an unequivocal unmet need. This is the first demonstration of clinical benefit for any systemic therapy for laBCC patients after HHI.

Clinically meaningful efficacy of cemiplimab in both BCC and CSCC is consistent with the shared clinical and molecular characteristics of these keratinocyte carcinomas (Nehal et al., 2018, N Engl J Med, 379:363-74). However, the ORR in this study (31%) is lower than that which was reported for advanced CSCC patients treated with cemiplimab (46%) (Rischin et al., 2020, J Immunother Cancer, 8:e000775). The BCC study is in the second-line (or greater) setting, whereas 66% (128/193) of advanced CSCC patients received cemiplimab in the first-line setting. In the second-line setting, the ORR for cemiplimab in the treatment of advanced CSCC was 42% (Rischin et al., 2020).

The kinetics of response to cemiplimab are slower in BCC than CSCC. Median time to response is 2 months in advanced CSCC patients treated with cemiplimab (Rischin et al., 2020), but is 4 months (range, 2 to 13) in this study. Responses to cemiplimab in both tumor types demonstrate durability, which is conclusively established in long-term follow-up in the CSCC study. Some PRs mature into CRs in CSCC patients. At the most recent update of the pivotal CSCC study, the group with the longest follow-up (Group 1, median follow-up 19 months) had a CR rate of 20%, compared with 7% at the time of primary analysis when median follow-up was 8 months (Rischin et al., 2020). Active follow-up of laBCC patients continues in this study, and some of the current PRs may evolve into CRs with continued follow-up.

Median TMB was higher in laBCC responders than in non-responders treated with cemiplimab. Not all patients with high TMB responded, raising the question of how some laBCCs with high TMB might evade an immune response. Downregulation of MHC-I expression is more common in BCC than in CSCC (Walters et al., 2010, Clin Cancer Res, 14:3562-70), prompting direct interrogation of this mechanism. We found that MHC-I expression was greatly reduced in non-responders compared with responders in the high TMB subset. Although downregulation of MHC-I occurs in a wide range of cancers, and there are case reports and retrospective studies describing potential worse clinical outcomes in tumors that downregulate it (Yoo et al., 2019, Sci Rep, 9:7680; Garrido et al., 2016, Curr Opin Immunol, 39:44-51), this is the first description of MHC-I downregulation as a mechanism of immune evasion during anti-PD1 therapy in a prospective clinical trial for any solid tumor type.

There is an emerging paradigm that clinical activity of immunotherapy is greatest when administered early in the natural history of cancers (Topalian et al., 2020, Science, 367). A combination of PD-1 blockade with HHIs in the first-line BCC setting may be appropriate for future clinical study. Preclinically, blockade of Smoothened signaling can inhibit formation of the immunological synapse (de la Roche et al., 2013, Science, 342:1247-50), and a pilot study of vismodegib+pembrolizumab did not suggest additive clinical activity (Chang et al., 2019, J Am Acad Dermatol, 80:564-66). Sequential therapy (HHI therapy, followed by PD-1 blockade) may be preferable, consistent with preliminary evidence that HHIs disrupt immune privilege in BCCs (Otsuka et al., 2015, J Dermatol Sci, 78:95-100).

In conclusion, the foregoing results show that cemiplimab is the first systemic therapy to demonstrate clinical benefit including durable responses in laBCC patients in the second-line (or greater) setting, after HHI therapy with a 31% ORR and an estimated 12-month probability of survival of 92.3%.

### Results (Group 1): Metastatic BCC (mBCC) Patients

Patient Characteristics: The results set forth herein are based on 28 patients with mBCC who were enrolled in the study, including patients with the opportunity to be followed for approximately 57 weeks to provide an ORR with 95% confidence interval (CI). Of the 28 mBCC patients, 82.1% were males and median age was 65.5 years (range 38-90). See Table 5.

TABLE 5
Patient Demographics and Baseline Characteristics

| Characteristics | mBCC (n = 28) | |
|---|---|---|
| Median age, years (range) | 65.5 | (38-90) |
| ≥65 years, n (%) | 15 | (53.6) |
| Male, n (%) | 23 | (82.1) |
| ECOG PS status, n (%) | | |
| 0 | 16 | (57.1) |
| 1 | 12 | (42.9) |
| Number of patients with prior HHI therapy, n (%) | | |
| Vismodegib | 28 | (100) |
| Sonidegib | 3 | (10.7) |
| Vismodegib + sonidegib | 3 | (10.7) |
| Reason for discontinuation of prior HHI, n (%)* | | |
| Progression of disease on HHI | 21 | (75.0) |
| Intolerant to prior HHI therapy | 10 | (35.7) |
| Intolerant to vismodegib | 11 | (39.3) |
| Intolerant to sonidegib | 2 | (7.1) |
| No better than stable disease after 9 months | 5 | (17.9) |
| on HHI therapy | | |
| Primary tumor site, n (%) | | |
| Head and neck | 11 | (39.3) |
| Trunk | 14 | (50.0) |
| Extremity | 2 | (7.1) |
| Anogenital | 1 | (3.6) |
| Metastatic status, n (%) | | |
| Distant only | 9 | (32.1) |
| Distant and nodal | 15 | (53.6) |
| Nodal only | 4 | (14.3) |
| Median duration of exposure, weeks (range) | 38.9 | (3.0-93.4) |
| Median number of doses administered (range) | 13 | (1-30) |

*Sum is >28 because some patients had more than one reason for discontinuation.

Clinical Efficacy: As summarized in Table 6, the ORR was 21.4% (95% CI, 8.3-41.0), with six patients showing a partial response. ORR per investigator assessment was 28.6% (95% CI, 13.2-48.7).

TABLE 6
Tumor Response and Duration of Response (DOR)

| n (%), unless otherwise stated | mBCC (n = 28) | |
|---|---|---|
| Best overall response | | |
| Overall response rate, % (95% CI) | 21.4 | (8.3-41.0) ^{a} |
| Complete response | 0 | |
| Partial response | 6 | (21.4) |
| Stable disease | 10 | (35.7) |
| Non-complete response/non-progressive disease | 3 | (10.7) |
| Progressive disease | 7 | (25.0) |
| Not evaluable ^{b} | 2 | (7.1) |
| Disease control rate, % (95% CI) ^{c} | 67.9 | (47.6-84.1) |
| Durable disease control rate, % (95% CI) ^{d} | 46.4 | (27.5-66.1) |
| Median (range) time to response, months ^{e} | 3.2 | (2.1-10.5) |
| Observed duration of response, range, months ^{e} | 9.0-23.0 | |
| ≥6 months | 6 | (100) |
| ≥12 months | 3 | (50.0) |
| Kaplan-Meier estimation of duration of response, | Not reached (9.0-NE) | |
| median (95% CI), months ^{e} | | |
| 6 months | 100 | (NE) |
| 12 months | 66.7 | (19.5-90.4) |
| Probability of progression-free survival, | | |
| % (95% CI) | | |
| 6 months | 58.1 | (37.1-74.3) |
| 12 months | 49.8 | (29.5-67.1) |

^{a} ORR per investigator was 28.6% (95% CI, 13.2-48.7).
^{b} Of the two patients who were not evaluable, one patient had no post-baseline assessment and one patient had no target or non-target lesions.
^{c} Defined as the proportion of patients with complete response, partial response, stable disease or non-partial response/non-progressive disease at the first evaluable tumor assessment, scheduled to occur at week 9 (defined as 56 days to account for visit windows in the protocol).
^{d} Defined as the proportion of patients without progressive disease for at least 182 days.
^{e} Data shown are for patients with response.

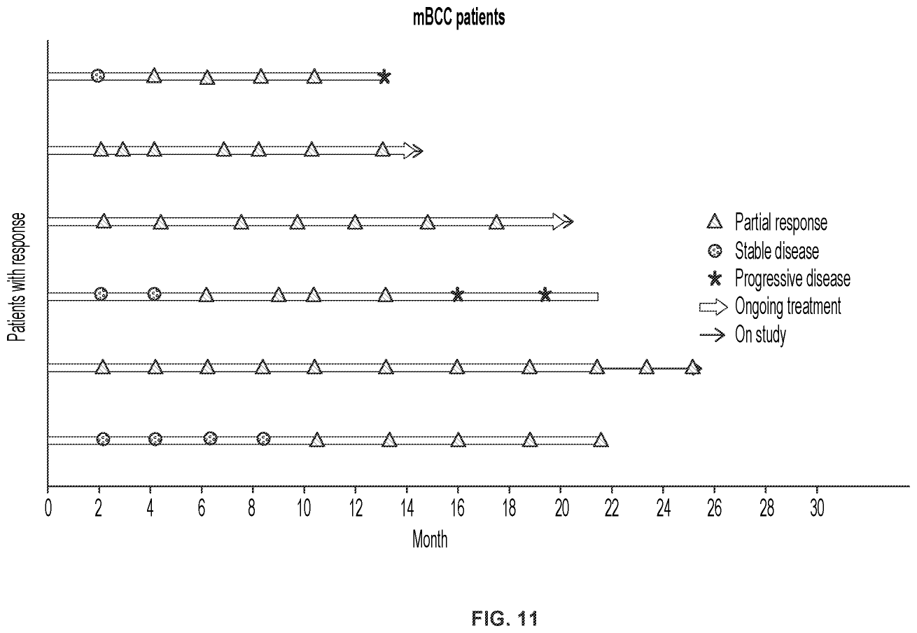

FIG. 11 provides swimmer plots that depict both time to response, and durability of responses of 6 patients with locally advanced BCC. In this figure, the closed arrow indicates the patient was still on treatment; and the open arrow indicates the patient was still on study. Each horizontal bar represents one patient. The disease control rate was 67.9% (95% CI, 47.6-84.1). The durable disease control rate was 46.4% (95% CI, 27.5-66.1). Among responders, median time to response per ICR was 3.2 months (range, 2.1-10.5). Observed duration of response (DOR) was 9-23 months. All six responses had observed duration of at least 8 months. duration of response (DOR) was 9-23 months. All six responses had observed duration of at least 8 months. Median DOR had not been reached.

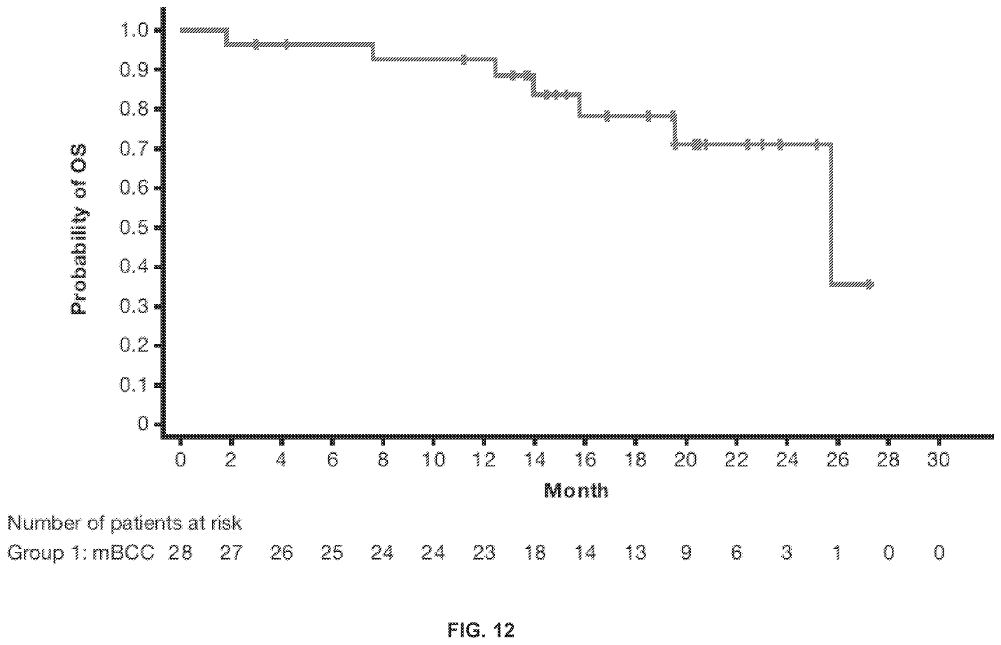

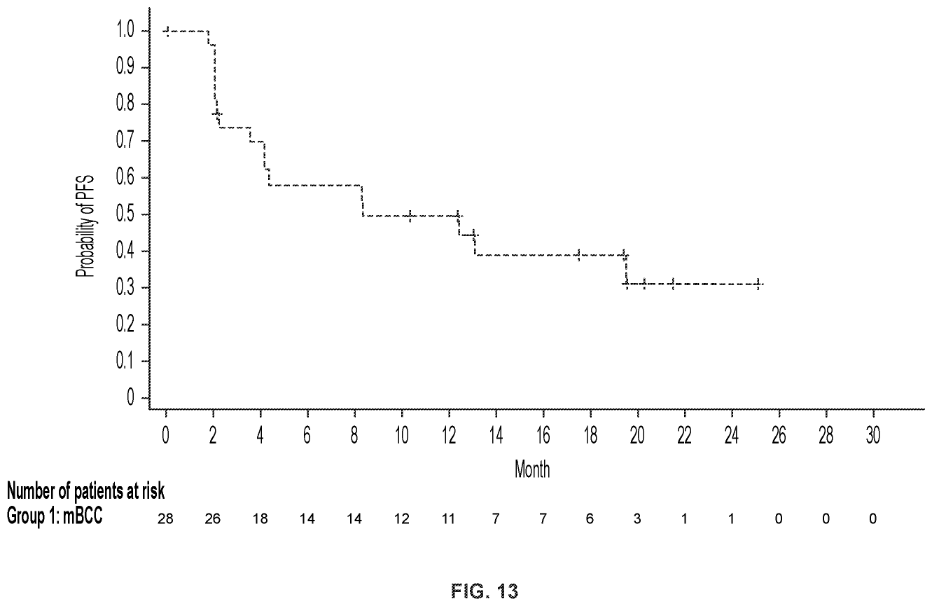

As shown in FIG. 12, Median Kaplan-Meier (KM) estimation of OS was 25.7 months (95% CI, 19.5—not evaluable [NE]). As shown in FIG. 13, median KM estimation of PFS was 8.3 months (95% CI, 3.6-19.5).

Treatment-emergent adverse events (TEAEs). TEAEs of any grade occurred in 26 (92.9%) patients. The most common TEAEs regardless of attribution were fatigue (50.0%), diarrhea (35.7%), pruritus (25.0%), and constipation (25.0%). Grade TEAEs were observed in 12 (42.9%) patients. Hypertension (n=2) was the only Grade TEAE regardless of attribution occurring in patients. TEAEs leading to death occurred in one (3.6%) patient who died from staphylococcal pneumonia, considered unrelated to study treatment. Treatment-related adverse events (TRAEs) of any grade occurred in 22 (78.6%) patients. The most common TRAEs regardless of attribution were fatigue (42.9%), pruritus (25.0%), and arthralgia (17.9%). Grade TRAEs were observed in five (17.9%) patients. Identified immune-related adverse events (irAEs) of any grade occurred in eight (28.6%) patients. The most common identified irAEs regardless of attribution were autoimmune hepatitis, colitis, hypothyroidism, and pneumonitis (each 7.1%). Grade identified irAES were observed in one (3.6%) patient. The only Grade identified irAE was colitis (3.6%).

In conclusion, the results presented here show that cemiplimab is the first agent to provide clinically meaningful anti-tumor activity, including durable responses, in patients with mBCC after progression or intolerance on HHI therapy. Cemiplimab is well tolerated and the safety profile is consistent with previous reports of cemiplimab in other tumor types. Combined with data from the laBCC cohort, these results confirm that cemiplimab is highly active in advanced BCC tumors. Further, it is expected that administration of cemiplimab leads to enhanced tumor regression in patients with other types of skin cancer tumors that exhibit threshold levels of TMB and MHC expression as discussed herein, including patients that experienced disease progression on HHI therapy or intolerance to HHI therapy, enabling such patients to achieve greater partial response and complete response, as well as significantly increased progression-free survival and overall response rate, as compared to patients with skin cancer tumors that do not exhibit the threshold levels of TMB and MHC set forth herein.

### REFERENCES

1. Puig S, Berrocal A. Management of high-risk and advanced basal cell carcinoma. Clin Transl Oncol 2015; 17:497-503.
2. Wu S, Han J, Li W-Q, Li T, Qureshi A A. Basal-cell carcinoma incidence and associated risk factors in US women and men. Am J Epidemiol 2013; 178:890-97.
3. Migden M R, Chang A L S, Dirix L, Stratigos A J, Lear J T. Emerging trends in the treatment of advanced basal cell carcinoma. Cancer Treat Rev 2018; 64:1-10.
4. Sekulic A, Migden M R, Oro A E, et al. Efficacy and safety of vismodegib in advanced basal-cell carcinoma. N Engl J Med 2012; 366:2171-79.
5. Migden M R, Guminski A, Gutzmer R, et al. Treatment with two different doses of sonidegib in patients with locally advanced or metastatic basal cell carcinoma (BOLT): a multicentre, randomised, double-blind phase 2 trial. Lancet Oncol 2015; 16:716-28.
6. Sekulic A, Migden M R, Basset-Seguin N, et al. Long-term safety and efficacy of vismodegib in patients with advanced basal cell carcinoma: final update of the pivotal ERIVANCE BCC study. BMC Cancer 2017; 17:332.
7. Dummer R, Guminksi A, Gutzmer R, et al. Long-term efficacy and safety of sonidegib in patients with advanced basal cell carcinoma: 42-month analysis of the phase II randomized, double-blind BOLT study. Br J Dermatol 2020; 182:1369-78.
8. Chang A L S, Oro A E. Initial assessment of tumor regrowth after vismodegib in advanced basal cell carcinoma. Arch Dermatol 2012; 148:1324-25.
9. Chalmers Z R, Connelly C F, Fabrizio D, et al. Analysis of 100,000 human cancer genomes reveals the landscape of tumor mutational burden. Genome Med 2017; 9:34.
10. Bonilla X, Parmentier L, King B, et al. Genomic analysis identifies new drivers and progression pathways in skin basal cell carcinoma. Nat Genet 2016; 48:398-406.
11. McGranahan N, Furness A J, Rosenthal R, et al. Clonal neoantigens elicit T cell immunoreactivity and sensitivity to immune checkpoint blockade. Science 2016; 351:1463-69.
12. Rizvi N A, Hellmann M D, Snyder A, et al. Cancer immunology. Mutational landscape determines sensitivity to PD-1 blockade in non-small cell lung cancer. Science 2015; 348:124-28.
13. Le D T, Durham J N, Smith K N, et al. Mismatch repair deficiency predicts response of solid tumors to PD-1 blockade. Science 2017; 357:409-13.
14. Euvrard S, Kanitakis J, Claudy A. Skin cancers after organ transplantation. N Engl J Med 2003; 348:1681-91.
15. Migden M R, Rischin D, Schmults C D, et al. PD-1 blockade with cemiplimab in advanced cutaneous squamous-cell carcinoma. N Engl J Med 2018; 379:341-51.
16. Migden M R, Khushalani N I, Chang A L S, et al. Cemiplimab in locally advanced cutaneous squamous cell carcinoma: results from an open-label, phase 2, single-arm trial. Lancet Oncol 2020; 21:294-305.
17. Rischin D, Migden M R, Lim A M, et al. Phase 2 study of cemiplimab in patients with metastatic cutaneous squamous cell carcinoma: primary analysis of fixed-dosing, long-term outcome of weight-based dosing. J Immunother Cancer 2020; 8:e000775.
18. Falchook G S, Leidner R, Stankevich E, et al. Responses of metastatic basal cell and cutaneous squamous cell carcinomas to anti-PD1 monoclonal antibody REGN2810. J Immunother Cancer 2016; 4:70.
19. Clopper C J, Pearson E S. The use of confidence or fiducial limits illustrated in the case of the binomial. Biometrika 1934; 26:404-13.
20. Barrios D M, Do M H, Phillips G S, et al. CME Part I: Immune checkpoint inhibitors to treat cutaneous malignancies. J Am Acad Dermatol 2020.
21. Sekulic A, Migden M R, Lewis K, et al. Pivotal ERIVANCE basal cell carcinoma (BCC) study: 12-month update of efficacy and safety of vismodegib in advanced BCC. J Am Acad Dermatol 2015; 72:1021-26 e8.
22. Basset-Seguin N, Hauschild A, Kunstfeld R, et al. Vismodegib in patients with advanced basal cell carcinoma: primary analysis of STEVIE, an international, open-label trial. Eur J Cancer 2017; 86:334-48.
23. Dreno B, Kunstfeld R, Hauschild A, et al. Two intermittent vismodegib dosing regimens in patients with multiple basal-cell carcinomas (MIKIE): a randomised, regimen-controlled, double-blind, phase 2 trial. Lancet Oncol 2017; 18:404-12.
24. Nehal K S, Bichakjian C K. Update on keratinocyte carcinomas. N Engl J Med 2018; 379:363-74.
25. Walter A, Barysch M J, Behnke S, et al. Cancer-testis antigens and immunosurveillance in human cutaneous squamous cell and basal cell carcinomas. Clin Cancer Res 2010; 14:3562-70.
26. Yoo S H, Keam B, Ock C Y, et al. Prognostic value of the association between MHC class I downregulation and PD-L1 upregulation in head and neck squamous cell carcinoma patients. Sci Rep 2019; 9:7680.
27. Garrido F, Aptsiauri N, Doorduijn E M, Garcia Lora A M, van Hall T. The urgent need to recover MHC class I in cancers for effective immunotherapy. Curr Opin Immunol 2016; 39:44-51.
28. Topalian S L, Taube J M, Pardoll D M. Neoadjuvant checkpoint blockade for cancer immunotherapy. Science 2020; 367.
29. de la Roche M, Ritter A T, Angus K L, et al. Hedgehog signaling controls T cell killing at the immunological synapse. Science 2013; 342:1247-50.
30. Chang A L S, Tran D C, Cannon J G D, et al. Pembrolizumab for advanced basal cell carcinoma: an investigator-initiated, proof-of-concept study. J Am Acad Dermatol 2019; 80:564-56.

31. Otsuka A, Levesque M P, Dummer R, Kabashima K. Hedgehog signaling in basal cell carcinoma. J Dermatol Sci 2015; 78:95-100.
The present disclosure is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

What is claimed is:

1. A method of treating or inhibiting the growth of a tumor, comprising:
(a) selecting a patient with cancer, wherein the patient has a tumor with a tumor mutation burden (TMB) of greater than or equal to 10 mutations/Mb, and wherein the patient does not exhibit downregulated major histocompatibility complex (MHC); and
(b) administering to the patient a therapeutically effective amount of programmed death 1 (PD-1) inhibitor.

2. The method of claim 1, wherein the cancer is skin cancer selected from basal cell carcinoma (BCC), cutaneous squamous cell carcinoma (CSCC), Merkel cell carcinoma, and melanoma.

3. The method of any one of claims 1-2, wherein the cancer is BCC.

4. The method of any one of claims 1-3, wherein the cancer is metastatic BCC or unresectable locally advanced BCC.

5. The method of any one of claims 1-4, wherein at least 35% of the tumor cells are positive for MHC expression.

6. The method of any one of claims 1-5, wherein the MHC is MHC-I.

7. The method of any one of claims 1-6, wherein the patient has experienced progression of disease on Hedgehog Inhibitor (HHI) therapy or was intolerant of prior HHI therapy.

8. The method according to any one of claims 1-7, wherein the PD-1 inhibitor is administered as a monotherapy.

9. The method according to any one of claims 1-8, wherein the administration of the PD-1 inhibitor promotes tumor regression, reduces tumor cell load, reduces tumor burden, and/or prevents tumor recurrence in the patient.

10. The method according to any one of claims 1-9, wherein the PD-1 inhibitor is administered in combination with a second therapeutic agent or therapy selected from radiation, surgery, a cancer vaccine, imiquimod, an anti-viral agent, photodynamic therapy, HHI therapy, a PD-L1 inhibitor, a LAG3 inhibitor, a cytotoxic CTLA-4 inhibitor, GITR agonist, a TIM3 inhibitor, a BTLA inhibitor, a TIGIT inhibitor, a CD38 inhibitor, a CD47 inhibitor, an IDO inhibitor, a CD28 activator, a VEGF antagonist, an Ang2 inhibitor, a TGF8 inhibitor, an EGFR inhibitor, an antibody to a tumor-specific antigen, a vaccine, a GM-CSF, an oncolytic virus, a cytotoxin, a chemotherapeutic agent, an IL-6R inhibitor, an IL-4R inhibitor, an IL-10 inhibitor, a cytokine, an antibody drug conjugate, an anti-inflammatory drug, and a dietary supplement.

11. The method according to any one of claims 1-10, wherein the PD-1 inhibitor is selected from an anti-PD-1 antibody or antigen-binding fragment thereof, an anti-PD-L1 antibody or antigen-binding fragment thereof, and an anti-PD-L2 antibody or antigen-binding fragment thereof.

12. The method according to any one of claims 1-11, wherein the PD-1 inhibitor is selected from an anti-PD-1 antibody or antigen-binding fragment thereof.

13. The method according to any one of claims 1-12, wherein the PD-1 inhibitor is an anti-PD-1 antibody or antigen-binding fragment thereof that comprises a heavy chain variable region (HCVR) comprising three heavy chain complementarity determining regions (CDRs) (HCDR1, HCDR2 and HCDR3) and a light chain variable region (LCVR) comprising three light chain CDRs (LCDR1, LCDR2 and LCDR3), wherein: HCDR1 has an amino acid sequence of SEQ ID NO: 3; HCDR2 has an amino acid sequence of SEQ ID NO: 4; HCDR3 has an amino acid sequence of SEQ ID NO: 5; LCDR1 has an amino acid sequence of SEQ ID NO: 6; LCDR2 has an amino acid sequence of SEQ ID NO: 7; and LCDR3 has an amino acid sequence of SEQ ID NO: 8.

14. The method according to claim 13, wherein the HCVR comprises an amino acid sequence of SEQ ID NO: 1.

15. The method according to claim 13, wherein the LCVR comprises an amino acid sequence of SEQ ID NO: 2.

16. The method according to claim 13, wherein the anti-PD-1 antibody or antigen-binding fragment thereof comprises an HCVR/LCVR amino acid sequence pair of SEQ ID NOs: 1/2.

17. The method according to any one of claims 13-16, wherein the anti-PD-1 antibody or antigen-binding fragment thereof comprises a heavy chain and a light chain, wherein the heavy chain has an amino acid sequence of SEQ ID NO: 9.

18. The method according to any one of claims 13-16, wherein the anti-PD-1 antibody comprises a heavy chain and a light chain, wherein the light chain has an amino acid sequence of SEQ ID NO: 10.

19. The method according to any one of claims 13-16, wherein the anti-PD-1 antibody comprises a heavy chain and a light chain, wherein the heavy chain has an amino acid sequence of SEQ ID NO: 9 and the light chain has an amino acid sequence of SEQ ID NO: 10.

20. The method according to any one of claims 1-12, wherein the PD-1 inhibitor is an anti-PD-1 antibody or antigen-binding fragment thereof comprising a HCVR with 90% sequence identity to SEQ ID NO: 1.

21. The method according to any one of claims 1-12, wherein the PD-1 inhibitor is an anti-PD-1 antibody or antigen-binding fragment thereof comprising a LCVR with 90% sequence identity to SEQ ID NO: 2.

22. The method according to any one of claims 1-12, wherein the PD-1 inhibitor is an anti-PD-1 antibody or antigen-binding fragment thereof comprising a HCVR with 90% sequence identity to SEQ ID NO: 1, and a LCVR with 90% sequence identity to SEQ ID NO: 2.

23. The method according to any one of claims 1-19, wherein the PD-1 inhibitor is cemiplimab or a bioequivalent thereof.

24. The method according to any one of claims 1-12, wherein the PD-1 inhibitor is an anti-PD-1 antibody selected from the group consisting of cemiplimab, nivolumab, pembrolizumab, pidilizumab, MED10608, BI 754091, PF-06801591, spartalizumab, camrelizumab, JNJ-63723283, and MCLA-134.

25. The method according to any one of claims 1-11, wherein the PD-1 inhibitor is an anti-PD-L1 antibody selected from the group consisting of REGN3504, avelumab, atezolizumab, durvalumab, MDX-1105, LY3300054, FAZ053, STI-1014, CX-072, KN035, and CK-301.

26. The method of any one of claims 1-25, wherein the PD-1 inhibitor is administered at a dose of 5 mg to 1500 mg.

27. The method of any one of claims 1-26, wherein the PD-1 inhibitor is administered at a dose of 200 mg, 250 mg, or 350 mg.

28. The method of any one of claims 1-25, wherein the PD-1 inhibitor is administered at a dose of 1 mg/kg to 20 mg/kg of the patient's body weight.

29. The method of any one of claims 1-25, wherein the PD-1 inhibitor is administered at a dose of 1 mg/kg, 3 mg/kg or 10 mg/kg of the patient's body weight.

30. The method of any one of claims 1-29, wherein the PD-1 inhibitor is administered as one or more doses, wherein each dose is administered two weeks, three weeks, four weeks, five weeks or six weeks after the immediately preceding dose.

31. The method according to any one of claims 1-30, wherein the PD-1 inhibitor is administered intravenously, subcutaneously, or intraperitoneally.

32. A kit comprising a programmed death 1 (PD-1) inhibitor in combination with written instructions for use of a therapeutically effective amount of the PD-1 inhibitor for treating or inhibiting the growth of a tumor in a patient with cancer, wherein the patient has a tumor with a tumor mutation burden (TMB) of greater than or equal to 10 mutations/Mb, and wherein the patient does not exhibit downregulated major histocompatibility complex (MHC).

33. A method of treating or inhibiting the growth of a tumor, comprising:
(a) selecting a patient with a basal cell carcinoma (BCC) tumor, wherein the patient has experienced progression of disease on Hedgehog Inhibitor (HHI) therapy or was intolerant of prior HHI therapy;
(b) collecting a biopsy of the tumor;
(c) measuring the tumor mutation burden (TMB) of the tumor biopsy;
(d) measuring the expression of major histocompatibility complex (MHC)-I in the tumor biopsy; and
(e) administering to the patient a therapeutically effective amount of programmed death 1 (PD-1) inhibitor if the tumor biopsy exhibits a TMB of greater than or equal to 10 mutations/Mb, and if at least 35% of the tumor biopsy cells are positive for MHC-I expression.

34. A method of selecting a patient with a basal cell carcinoma (BCC) tumor for treatment with a programmed death 1 (PD-1) inhibitor, comprising:
(a) collecting a biopsy of the BCC tumor;
(b) measuring the tumor mutation burden (TMB) of the tumor biopsy;
(c) measuring the expression of major histocompatibility complex (MHC)-I in the tumor biopsy; and
(d) selecting the patient for treatment with a PD-1 inhibitor if the tumor biopsy has a TMB of greater than or equal to 10 mutations/Mb, and a positive MHC-I expression in at least 35% of tumor cells.
